(19) United States
(12) Patent Application Publication
(10) Pub. No.: US 2018/0140750 A1
(43) Pub. Date: May 24, 2018

(54) MEDICAL INSTRUMENT

(71) Applicant: FUJIFILM Corporation, Tokyo (JP)

(72) Inventors: Michihiro SHIBATA, Ashigara-kami-gun (JP); Hideo NAGASAKI, Ashigara-kami-gun (JP); Fumito NARIYUKI, Ashigara-kami-gun (JP); Setsuko SHIRATSUCHI, Ashigara-kami-gun (JP); Shigeaki OHTANI, Ashigara-kami-gun (JP); Norihiro OMAE, Ashigara-kami-gun (JP); Haruyasu NAKATSUGAWA, Ashigara-kami-gun (JP)

(73) Assignee: FUJIFILM Corporation, Tokyo (JP)

(21) Appl. No.: 15/875,001

(22) Filed: Jan. 19, 2018

Related U.S. Application Data
(63) Continuation of application No. PCT/JP2016/062378, filed on Apr. 19, 2016.

(30) Foreign Application Priority Data
Jul. 21, 2015 (JP) 2015-143630

(51) Int. Cl. A61L 31/14; A61B 90/70; A61L 31/08

(52) U.S. Cl. CPC A61L 31/14; A61B 90/70; A61B 1/127; A61L 2202/24; A61L 2202/11; A61L 31/08

(57) ABSTRACT

A medical instrument of the present invention includes a hydrophilic processed portion on at least a portion of an outer surface of the instrument. The hydrophilic processed portion contains a hydrophilic polymer and an antibacterial agent, and a water contact angle of a surface of the hydrophilic processed portion is equal to or smaller than 30°. Provided that a silver content per unit area in the hydrophilic processed portion is P, and a silver ion amount per unit area measured by an extraction test described below is Q, P and Q satisfy the relationships of Formula (1) and Formula (2) shown below. Accordingly, the medical instrument has excellent antifogging properties and antibacterial properties and excellently retains the antibacterial properties.

6.0≤P/Q Formula (1)

15.05≤Q Formula (2)

The unit of P is ng/cm², and the unit of Q is ng/cm².

### CROSS-REFERENCE TO RELATED APPLICATIONS

This application is a Continuation of PCT International Application No. PCT/JP2016/062378 filed on Apr. 19, 2016, which claims priority under 35 U.S.C. § 119(a) to Japanese Patent Application No. 2015-143630 filed on Jul. 21, 2015. Each of the above applications is hereby expressly incorporated by reference, in its entirety, into the present application.

### BACKGROUND OF THE INVENTION

### 1. Field of the Invention

The present invention relates to a medical instrument including a hydrophilic processed portion on at least a portion of a surface of the instrument.

### 2. Description of the Related Art

Many of medical instruments used in the medical field continuously come into contact with a plurality of patients. In a case where the body fluid of the patients and the like adhere to these medical instruments and are insufficiently cleaned or washed, a pathogenic viral infection and the like may be transmitted between the patients or between the patient and a radiographer. In order to prevent such infection, the surface of the medical instruments is sterilized using a disinfectant solution such as an aqueous ethanol solution or an aqueous sodium hypochlorite solution, regularly or whenever the instruments are used for a new patient in a case where there is a high risk of infection.

For example, in a case where a radiographic imaging device, especially, a portable radiographic imaging device is used in an operating room, an emergency room, or the like, blood or vomit containing infectious viruses or bacteria, body fluid, sebum, and the like adhere to the device in some cases. Furthermore, in a mammography device, lipstick or sebum from a patient adheres to a face guard portion, or oozing breast milk, blood resulting from hemorrhage at the time of biopsy (mammotome biopsy), or sebum adheres to a breast support or a breast compression plate. These types of contaminants from patients are not easily wiped off. Particularly, in some cases, because the contaminants stick to the surface of the device, it takes time to wipe the contaminants off.

As solutions to these problems, the following techniques are suggested.

For example, JP2012-132703A discloses a technique of mounting a buffer material, which has undergone waterproofing processing, on an outer surface of a medical instrument. This technique has an advantage of hindering the adherence of contaminants.

Furthermore, JP2012-123297A suggests a method of coating a medical instrument with a photocatalyst (for example, TiO₂) that shows a bactericidal action.

In addition, from the viewpoint of a countermeasure for infection, JP1997-131389A (JP-H09-131389A) or JP2002-337277A suggests a technique for reducing the bacterial multiplication or the risk of the infection of diseases by providing an antibacterial layer on the surface of various medical instruments including a radiographic imaging device.

JP1997-131389A (JP-H09-131389A) discloses a slow-release antibacterial material formed of a silver-based antibacterial agent in which a silver complex is supported on a silica gel support, silica gel, and an organic binder.

JP2002-337277A discloses a front panel formed of a scratch-resistant film, which includes an antibacterial agent formed of a silver-containing phosphate double salt, and a substrate.

In some cases, due to humidity, dew condensation occurs on the surface of the medical instruments used in the medical field, the display screen of the instruments becomes foggy, and the visibility deteriorates.

For example, some of the cassettes used in radiographic imaging devices include an LED display portion, which is for checking the position of a panel by the light emitted from a light emitting portion or for preventing the mounting of a wrong panel by the difference of color of the emitted light, or a display portion showing a battery level. In a case where the display portion is foggy, the visibility of light decreases, and the workflow deteriorates.

As a solution to the problem relating to antifogging properties, JP2002-113053A suggests a method in which in a plastic hood, that is, in an incubator hood made of plastic, at least a portion of the inner surface of the hood is coated with a photocatalyst such that antifogging properties and antibacterial properties are imparted, and that the dew condensation and the multiplication of fungi and bacteria are prevented from occurring on the inner surface of the plastic hood.

### SUMMARY OF THE INVENTION

In view of sterilization, by the technique described in JP2012-132703A, the surface of medical instruments exhibits low wettability with respect to a disinfectant solution. That is, because the disinfectant solution is repelled, it is not easy for the disinfectant solution to stay for a period of time on the outer surface of medical instruments, and as a result, the sterilization effect is highly unlikely to be sufficiently exerted on bacteria.

Furthermore, in order for the photocatalyst described in JP2012-123297A or JP2002-113053A to exhibit a sterilizing performance, hydrophilicity, or antifogging properties, the photocatalyst must be irradiated with light. Therefore, the device is not suitable for being used in various medical environments including a dark place.

In recent years, in order to enable medical devices to be more frequently used, the medical devices have been required to exhibit an antibacterial action within a shorter period of time. Furthermore, the medical devices have been required to be able to retain the antibacterial action for a longer period of time. That is, there is a demand for medical instruments which exhibit the antibacterial action within a short period of time and can retain the antibacterial properties for a long time.

However, as a result of examining the substrate with an antibacterial layer described in JP1997-131389A (JP-H09-131389A) and JP2002-337277A, the inventors of the present invention have found that the substrate does not sufficiently satisfy the aforementioned demand and needs to be further improved.

The present invention is for solving the aforementioned problems, and an object thereof is to provide a medical instrument which has excellent antifogging properties and antibacterial properties and excellently retains the antibacterial properties.

In order to achieve the aforementioned object, a medical instrument of a first aspect of the present invention comprises a hydrophilic processed portion on at least a portion of an outer surface of the instrument, in which the hydrophilic processed portion contains a hydrophilic polymer and an antibacterial agent, a water contact angle of a surface of the hydrophilic processed portion is equal to or smaller than 30°, and provided that a silver content per unit area in the hydrophilic processed portion is P and a silver ion amount per unit area measured by an extraction test described below is Q, P and Q satisfy relationships of Formula (1) and Formula (2).

6.0≤P/Q Formula (1)

15.0≤Q Formula (2)

Herein, a unit of P is ng/cm², and a unit of Q is ng/cm².

The extraction test is a test in which a 1/500 normal nutrient broth medium specified in JIS Z 2801:2010 is used as an extractant; a temperature of the extractant is controlled within a range of 35±1° C.; the extractant is brought into contact with the hydrophilic processed portion for 1 hour; the silver ion amount extracted into the extractant is measured; and the obtained value is divided by a contact area between the hydrophilic processed portion and the extractant, thereby obtaining the silver ion amount Q per unit area.

Herein, Q preferably satisfies a relationship of Formula (3).

15.0≤Q≤25.0 Formula (3)

The antibacterial agent preferably includes a first antibacterial agent containing silver and a second antibacterial agent containing silver different from the first antibacterial agent.

The first antibacterial agent preferably contains silver and one support selected from the group consisting of calcium zinc phosphate and calcium phosphate, and the second antibacterial agent preferably contains silver and a support formed of zeolite.

A medical instrument of a second aspect of the present invention comprises a hydrophilic processed portion on at least a portion of an outer surface of the instrument, in which the hydrophilic processed portion contains a hydrophilic polymer and at least one kind of antibacterial agent, a water contact angle of a surface of the hydrophilic processed portion is equal to or smaller than 30°, the antibacterial agent contains silver, and a silver ion amount per unit area measured by an extraction test described below is 15 to 50 ng/cm².

In the extraction test, a 1/500 normal nutrient broth medium specified in JIS Z 2801:2010 is used as an extractant; a temperature of the extractant is controlled within a range of 35±1° C.; the extractant is brought into contact with the hydrophilic processed portion for 1 hour; the silver ion amount extracted into the extractant is measured; and the obtained value is divided by a contact area between the hydrophilic processed portion and the extractant, thereby obtaining the silver ion amount per unit area. A unit of the silver ion amount is ng, a unit of the contact area is cm², and a unit of the silver ion amount per unit area is ng/cm².

Herein, the antibacterial agent is preferably a silver-supporting support containing a support and a silver supported on the support.

A medical instrument of a third aspect of the present invention comprises a hydrophilic processed portion on at least a portion of an outer surface of the instrument, in which the hydrophilic processed portion contains a hydrophilic polymer, an antibacterial agent containing silver, and a porous support which can adsorb silver ions, a water contact angle of a surface of the hydrophilic processed portion is equal to or smaller than 30°, and provided that an average particle diameter of the antibacterial agent is Da, an average particle diameter of the porous support is Db, and an average thickness of the hydrophilic processed portion is T, Da, Db, and T satisfy relationships shown below.

T/Da>3.0 Formula (4)

T/Db≤3.0 Formula (5)

A unit of Da, Db, and T is μm.

It is preferable that silver is supported on the porous support.

Da and Db preferably satisfy a relationship shown below.

Db/Da≤3.5 Formula (6)

A content of the porous support with respect to a total mass of the hydrophilic processed portion is preferably equal to or smaller than 0.5% by mass.

The antibacterial agent preferably contains silver and one support selected from the group consisting of calcium zinc phosphate and calcium phosphate, and the porous support preferably contains silver and a support formed of zeolite.

The medical instruments of the first to third aspects preferably satisfy the following conditions.

A surface roughness Ra of the surface of the hydrophilic processed portion is 2 to 15 μm.

The hydrophilic processed portion contains lubricant particles having an average particle diameter of 6 to 10 μm.

The average thickness of the hydrophilic processed portion is 1 to 10 μm.

A content of the antibacterial agent with respect to the total mass of the hydrophilic processed portion is 0.001% to 5% by mass.

The antibacterial agent contains at least one kind of particles selected from the group consisting of ceramic particles supporting silver and silver particles.

The medical instruments of the first to third aspects are preferably a radiographic imaging device.

In the radiographic imaging device, the hydrophilic processed portion is preferably provided on at least a surface that comes into contact with a subject at the time of imaging.

The radiographic imaging device is preferably a portable radiographic imaging device, and the hydrophilic processed portion is preferably provided on at least a surface on a side irradiated with radiation in a housing of the portable radiographic imaging device.

The hydrophilic processed portion is preferably further provided in a rear surface portion of the housing and/or a lateral surface portion of the housing.

The portable radiographic imaging device as the radiographic imaging device preferably has a housing body in the form of a cylinder having at least one open end, and the housing body preferably constitutes the housing by closing the one open end with a cap member.

In the portable radiographic imaging device as the radiographic imaging device, at least a portion of the rear surface portion of the housing and at least a portion of the lateral surface portion of the housing are preferably integrally formed.

The radiographic imaging device is preferably a mammography device, and the hydrophilic processed portion is preferably provided on at least a surface coming into contact with a subject at the time of imaging within a surface of a face guard portion.

In the mammography device, the hydrophilic processed portion is preferably provided on at least a surface coming into contact with a subject within a surface of a breast support or on at least a surface coming into contact with a subject within a surface of a breast compression plate.

The medical instrument is preferably a medical mask.

The medical instrument is preferably a medical goggle.

The medical instrument is preferably a transparent sterile medical bag.

According to the present invention, a hydrophilic processed portion having excellent antifogging properties and antibacterial properties is provided on the surface of a medical instrument. Therefore, the present invention can provide a medical instrument in which the fogging of the surface of the instrument can be prevented or inhibited and the bacterial multiplication can be prevented or inhibited.

### BRIEF DESCRIPTION OF THE DRAWINGS

FIG. 1 a perspective cross-sectional view showing a portion of a portable radiographic imaging device as a radiographic imaging device according to Embodiment 1 of the present invention.

FIG. 2 is a schematic longitudinal cross-sectional view of the portable radiographic imaging device shown in FIG. 1.

FIG. 3A is an enlarged longitudinal cross-sectional view in which an end portion of the portable radiographic imaging device shown in FIG. 2 is enlarged, and FIG. 3B is a perspective view schematically showing the entirety of the portable radiographic imaging device shown in FIG. 1 from a rear surface side.

FIG. 4 is a schematic cross-sectional view of an example of a substrate with a hydrophilic processed portion according to Embodiment 1 of the present invention.

FIGS. 5A and 5B are schematic cross-sectional views of an example of a hydrophilic processed sheet used for providing a hydrophilic processed portion on a surface of the portable radiographic imaging device shown in FIG. 1.

FIGS. 6A, 6B, and 6C are schematic cross-sectional views of an example of a base with a hydrophilic processed antibacterial film that is provided directly on the surface of the portable radiographic imaging device shown in FIG. 1.

FIG. 7 is a schematic cross-sectional view of an example of a substrate with a hydrophilic processed portion according to Embodiment 2 of the present invention.

FIG. 8 is a schematic cross-sectional view of an example of a substrate with a hydrophilic processed portion according to Embodiment 3 of the present invention.

FIGS. 9A and 9B are views for illustrating a mechanism by which the substrate with a hydrophilic processed portion according to Embodiment 3 of the present invention exhibits antibacterial properties.

FIG. 10 is a schematic longitudinal cross-sectional view of a portable radiographic imaging device as a radiographic imaging device according to Embodiment 4 of the present invention.

FIG. 11 is a schematic perspective view showing main portions of a mammography device as a radiographic imaging device according to Embodiment 5 of the present invention.

FIG. 12 is a schematic perspective view showing main portions of a radiographic imaging device for upright radiography as a radiographic imaging device according to Embodiment 6 of the present invention.

FIG. 13 is a perspective view of a medical mask according to Embodiment 7 of the present invention.

FIG. 14 is a perspective view of a medical goggle according to Embodiment 8 of the present invention.

FIG. 15 is a perspective view of a transparent sterile medical bag according to Embodiment 9 of the present invention.

### DESCRIPTION OF THE PREFERRED EMBODIMENTS

Hereinafter, a medical instrument according to the present invention will be specifically described based on suitable embodiments with reference to the attached drawings.

In the present specification, a range of numerical values described using “to” means a range including the numerical values listed before and after “to” as a lower limit and an upper limit respectively.

### Embodiment 1

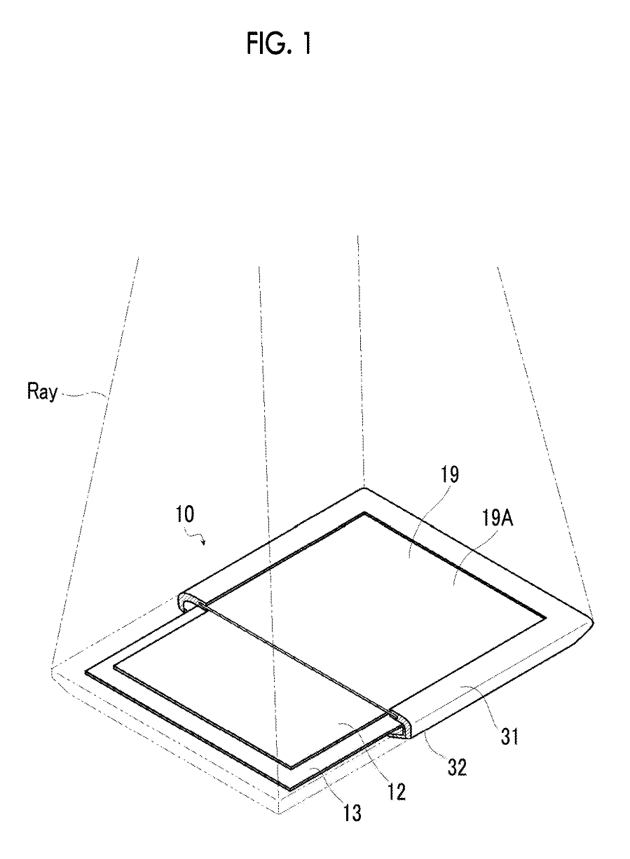

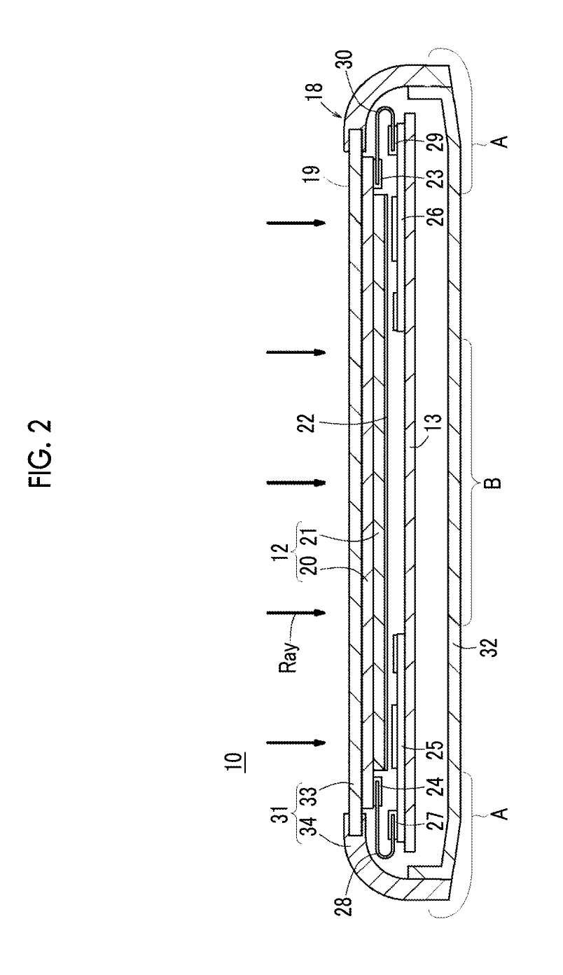

FIG. 1 is a perspective cross-sectional view showing a portion of a portable radiographic imaging device 10 (so-called electronic cassette) according to Embodiment 1. The portable radiographic imaging device 10 is a type of radiographic imaging device. FIG. 2 is a schematic longitudinal cross-sectional view of the portable radiographic imaging device 10. In the portable radiographic imaging device 10, on the inside of a housing 18, from an irradiation surface 19 side irradiated with radiation Ray, a radiation detector 12, which detects the radiation Ray transmitted through a patient not shown in the drawing, and a control board 13 which will be described later are provided in this order. Within the irradiation surface 19, a region in which a radiograph is imaged by the radiation detector 12 is an imaging region 19A.

The radiation detector 12 is constituted with a thin film transistor (TFT) active matrix board (hereinafter, referred to as a TFT board) 20 and a scintillator 21 formed of gadolinium oxysulfide (GOS), cesium iodide (CsI), or the like and is bonded to the surface of the TFT board 20. In order to prevent the generated light from leaking to the outside, the TFT board 20 may have a light screen element 22, which blocks the generated light, on a surface that is on the side opposite to the surface to which the scintillator 21 is bonded.

In the radiation detector 12, the radiated radiation Ray such as an X-ray is converted into light by the scintillator 21. The generated light comes into sensor portions provided in the TFT board 20. The sensor portions receive the light generated from the scintillator 21 and accumulate a charge. Each of the sensor portions is provided with a TFT switch. In a case where the TFT switch is turned ON, according to the amount of charge accumulated in the sensor portion, an electric signal (image signal) showing a radiograph flows in a signal line.

One end of the radiation detector 12 in a signal wiring direction is provided with a plurality of connectors 23 for wire connection in a line, and the other end of the radiation detector 12 in a scanning wiring direction is provided with a plurality of connectors 24 in a line. The connectors 23 are connected to signal wiring, and the connectors 24 are connected to scanning wiring.

The control board 13 includes a scan signal control circuit 25 and a signal detection circuit 26. The scan signal control circuit 25 is provided with a connector 27, and the connector 27 is electrically connected to one end of a flexible cable 28. The other end of the flexible cable 28 is electrically connected to the connectors 24. By this constitution, the scan signal control circuit 25 can output control signals to each scanning wiring for turning the TFT switch ON/OFF.

The signal detection circuit 26 is provided with a plurality of connectors 29, and the connectors 29 are electrically connected to one end of a flexible cable 30. The other end of the flexible cable 30 is electrically connected to the connectors 23. The signal detection circuit 26 has a built-in amplification circuit, which is for amplifying the input electric signals, for each signal wiring. By this constitution, the signal detection circuit 26 detects the electric signal input from each signal wiring by amplifying the electric signal through the amplification circuit. In this way, the signal detection circuit 26 detects the amount of charge accumulated in each of the sensor portions as the information on the respective pixels constituting an image.

The housing 18 has the built-in control board 13 which is in the form of a rectangular flat plate and performs various types of control such as the control of the imaging operation of the radiation detector 12 or the control of the communication with external devices as if overlapping the radiation detector 12 as shown in FIG. 2.

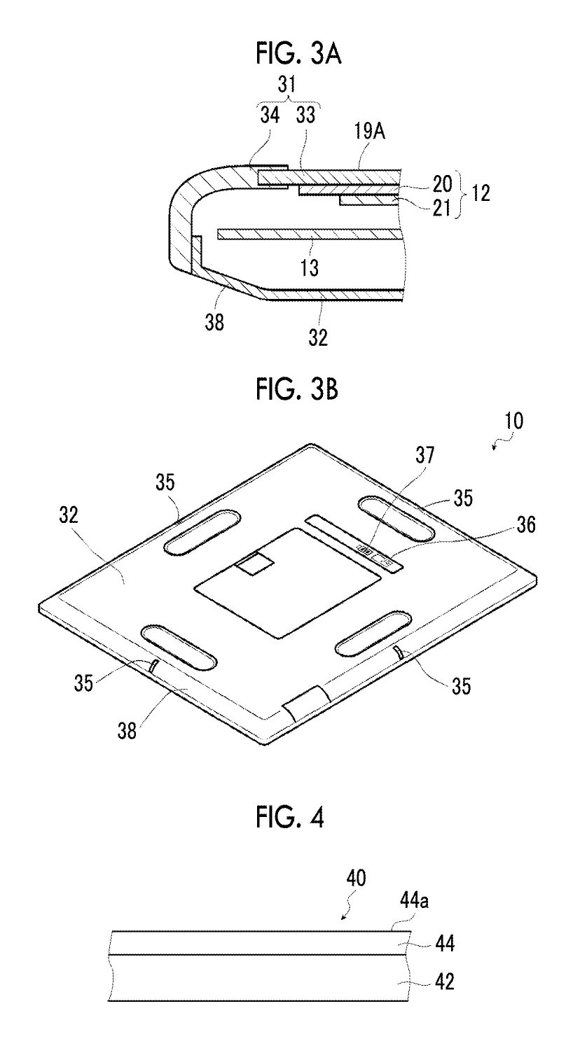

In the housing 18, as shown in FIG. 3A, a front panel 31, which is disposed on a front surface side irradiated with the radiation Ray, in other words, on the side coming into contact with a subject, and a back panel 32 which is disposed on a side opposite to the subject are provided in a state of facing each other. The front panel 31 is constituted with a top panel 33 and a holding portion 34 that holds the top panel 33. A surface of the top panel 33 on the back panel 32 side is provided with the radiation detector 12. At both ends of FIG. 2 in a horizontal direction, the holding portion 34 curves toward the back panel 32 side so as to form a portion of the lateral surface portion. Furthermore, at both ends of FIG. 3A in a horizontal direction, the back panel 32 curves toward the front panel 31 side so as to form an inclined portion 38 as a portion of the lateral surface portion. That is, the rear surface portion of the housing 18 and the inclined portion 38 as a portion of the lateral surface portion are integrally formed. Herein, it is not necessary for the rear surface portion, but only a portion of the lateral surface portion, to be integrally formed. The entirety of the lateral surface portion and the rear surface portion may be integrally formed. At this time, the number of seams of the housing can be reduced, and hence wiping properties are improved.

Herein, the lateral surface portion includes a portion of the holding portion 34 curving toward the back panel 32 side and the inclined portion 38 of the back panel 32. The rear surface portion refers to a portion of the back panel 32 that does not curve toward the front panel 31 side, that is, a portion of the back panel 32 excluding the inclined portion 38.

As shown in FIG. 3B, the back panel 32 includes, in the inclined portion, an LED lamp 35 which makes it possible to prevent the mounting of a wrong panel in a case where a plurality of panels are used and to easily check the center position of a panel at the time of imaging. Furthermore, the rear surface portion of the back panel 32 is provided with a counting display portion 36 displaying the total number of sheets of images captured or a battery level display portion 37. The LED lamp 35 is disposed at the central portion of the inclined portion 38 of each side forming the back panel 32.

In the present embodiment, the top panel 33 is formed of carbon. Accordingly, it is possible to secure hardness while inhibiting the absorption of the radiation Ray. The holding portion 34 and the back panel 32 are formed of an ABS resin.

Within the top panel 33, a region in which a radiograph is imaged by the radiation detector 12 is the imaging region 19A.

In the aforementioned constitution, at least the irradiation surface 19, which comes into contact with a subject (not shown in the drawing) such as a patient at the time of imaging, the LED lamp 35, the counting display portion 36, and the battery level display portion 37 are provided with the hydrophilic processed portion. In addition, it goes without saying that a portion which can come into contact with a radiographer, for example, the entirety of the outer surface may be provided with the hydrophilic processed portion.

In the present invention, in a case where the portable radiographic imaging device (electronic cassette) 10 is inserted under a subject such as a patient, the hydrophilic processed portion provided on the irradiation surface 19 and the like becomes a surface that comes into contact with the subject. Therefore, in order to make the hydrophilic processed portion slide easily, a dot shape or a mesh shape may be formed thereon in advance.

Furthermore, for example, it is preferable to perform a hydrophilic treatment on the surface of the front panel 31 on the irradiation surface 19 side such that the hydrophilic processed portion is provided on the irradiation surface 19. In addition, it is preferable to perform a hydrophilic treatment on the outer surface of the LED lamp 35, the counting display portion 36, and the battery level display portion 37 of the back panel 32 on the side opposite to the irradiation surface 19 (on the side opposite to a subject) such that the hydrophilic processed portion is provided thereon. Furthermore, it is preferable to perform a waterproofing treatment on the outer surface of the back panel 32, particularly, on the portion indicated by the reference A on both sides of the back panel 32. That is, it is preferable to perform a waterproofing treatment on a portion A of the outer surface from the seam between the back panel 32 and the holding portion 34 of the front panel 31 constituting a portion of the lateral surface portion of the housing 18 to the region of the end of the back panel 32 constituting the rear surface portion of the housing 18, such that a waterproofing-processed portion is provided in the portion A.

In a case where the waterproofing-processed portion is provided on the outer surface (the portion A on both sides) of the back panel 32, the portable radiographic imaging device (electronic cassette) 10 can be easily inserted under the subject such as a patient.

In a case where a radiographic imaging device has a structure in which corners of the electronic cassette incline (curve) similarly to the holding portion 34 of the housing 18 of the portable radiographic imaging device (electronic cassette) 10 shown in FIG. 2, a contaminant may drip, and hence the periphery of the electronic cassette may be contaminated. However, in the present invention, the hydrophilic processed portion is provided on the surface of the front panel 31 on the irradiation surface 19 side, and a waterproofing-processed portion is provided on the outer surface (the portion A on both sides) of the back panel 32. Therefore, the front panel 31 exhibits excellent wettability, a contaminant can be prevented from dripping from the holding portion 34 of the front panel 31, and the diffusion of the contaminant can be prevented.

In addition, in a case where embossing processing is performed on a central portion B of the outer surface of the back panel 32 constituting the rear surface portion of the housing 18 such that the central portion B has an embossed structure, the electronic cassette 10 can be easily inserted under a subject such as a patient.

(Substrate)

In the present embodiment, the members provided with the hydrophilic processed portion, that is, the top panel 33 is formed of carbon, and the holding portion 34 and the back panel 32 are formed of an ABS resin. However, the type of material forming such members is not particularly limited as long as the members play the role of supporting the hydrophilic processed portion. For example, it is possible to use a metal, glass, ceramics, plastic (resin), and the like. Among these, in view of handleability, plastic is preferable. In other words, a resin substrate is preferable.

The shape of the substrate is not particularly limited, and it is possible to use substrates having a plate shape, a film shape, a sheet shape, a tube shape, a fiber shape, or a particle shape. Furthermore, the surface of the substrate may be flat, concave, or convex.

The hydrophilic processed portion contains at least a hydrophilic polymer and an antibacterial agent.

Hereinafter, the materials contained in the hydrophilic processed portion will be specifically described.

(Hydrophilic Polymer)

The hydrophilic polymer is a polymer having a hydrophilic group.

The type of the hydrophilic group is not particularly limited, and examples thereof include a polyoxyalkylene group (for example, a polyoxyethylene group, a polyoxypropylene group, or a polyoxyalkylene group in which an oxyetylene group and an oxypropylene group are bonded to each other in the form of a block copolymer or a random copolymer), an amino group, a carboxyl group, an alkali metal salt of a carboxyl group, a hydroxyl group, an alkoxy group, an amide group, a carbamoyl group, a sulfonamide group, a sulfamoyl group, a sulfonic acid group, an alkali metal salt of a sulfonic acid group, and the like.

The structure of a main chain of the hydrophilic polymer is not particularly limited, and examples thereof include polyurethane, a poly(meth)acrylic acid ester, polystyrene, polyester, polyamide, polyimide, polyurea, and the like.

Conceptually, the poly(meth)acrylic acid ester includes both of a polyacrylic acid ester and a polymethacrylic acid ester.

As one of the suitable aspects of the hydrophilic polymer, a polymer can be exemplified which is obtained by polymerizing a monomer having the aforementioned hydrophilic group.

The monomer having a hydrophilic group means a compound having the aforementioned hydrophilic group and a polymerizable group. The definition of the hydrophilic group is as described above.

The number of hydrophilic groups in the monomer having a hydrophilic group is not particularly limited. In view of making the hydrophilic processed portion exhibit stronger hydrophilicity, the number of the hydrophilic groups is preferably equal to or greater than 2, more preferably 2 to 6, and even more preferably 2 or 3.

The type of the polymerizable group is not particularly limited, and examples thereof include a radically polymerizable group, a cationically polymerizable group, an anionically polymerizable group, and the like. Examples of the radically polymerizable group include a (meth)acryloyl group, an acrylamide group, a vinyl group, a styryl group, an allyl group, and the like. Examples of the cationically polymerizable group include a vinyl ether group, an oxiranyl group, an oxetanyl group, and the like. Among these, a (meth)acryloyl group is preferable.

Conceptually, the (meth)acryloyl group includes both of an acryloyl group and a methacryloyl group.

The number of polymerizable groups in the monomer having a hydrophilic group is not particularly limited. In view of further improving the mechanical strength of the obtained hydrophilic processed portion, the number of polymerizable groups is preferably equal to or greater than 2, more preferably 2 to 6, and even more preferably 2 or 3.

As one of the suitable aspects of the monomer having a hydrophilic group, a compound represented by Formula (A) can be exemplified.

(R₁₄₋ₙCL₁-L₂-R₂)ₙ Formula (A)

In Formula (A), R₁ represents a substituent. The type of the substituent is not particularly limited. Examples of the substituent include known substituents such as a hydrocarbon group (for example, an alkyl group or an aryl group) which may have a heteroatom, the aforementioned hydrophilic group, and the like.

R₂ represents a polymerizable group. The definition of the polymerizable group is as described above.

L₁ represents a single bond or a divalent linking group. The type of the divalent linking group is not particularly limited, and examples thereof include —O—, —CO—, —NH—, —CO—NH—, —COO—, —O—COO—, an alkylene group, an arylene group, a heteroaryl group, and a combination of these.

L₂ represents a polyoxyalkylene group. The polyoxyalkylene group means a group represented by Formula (B).

*—(OR₃)ₘ—* Formula (B)

In Formula (B), R₃ represents an alkylene group (for example, an ethylene group or a propylene group). m represents an integer of equal to or greater than 2. m is preferably 2 to 10, and more preferably 2 to 6. * represents a binding position.

n represents an integer of 1 to 4.

In order to obtain a hydrophilic polymer, the aforementioned monomer having a hydrophilic group may be used in combination with other monomers. That is, a hydrophilic polymer may be used which is obtained by copolymerizing the monomer having a hydrophilic group and other monomers (monomers other than the monomer having a hydrophilic group).

The type of other monomers is not particularly limited, and known monomers can be appropriately used as long as they have a polymerizable group. The definition of the polymerizable group is as described above.

Among the monomers, in view of further improving the mechanical strength of the hydrophilic processed portion, a polyfunctional monomer having two or more polymerizable groups is preferable. The polyfunctional monomer acts as a so-called cross-linking agent.

The number of polymerizable groups contained in the polyfunctional monomer is not particularly limited. In view of further improving the mechanical strength of the hydrophilic processed portion and in view of handleability, the number of polymerizable groups is preferably 2 to 10 and more preferably 2 to 6.

Examples of the polyfunctional monomer include trimethylolpropane triacrylate, tetramethylolmethane tetraacrylate, dipentaerythritol hexaacrylate, and pentaerythritol tetraacrylate.

A mixing ratio (mass of hydrophilic monomer/mass of other monomers) of the hydrophilic monomer to other monomers (particularly, polyfunctional monomers) is not particularly limited. In view of making it easy to control the hydrophilicity of the hydrophilic processed portion, the mixing ratio is preferably 0.01 to 10 and more preferably 0.1 to 10.

It is preferable that the hydrophilic processed portion contains the aforementioned hydrophilic polymer as a main component. Herein, the main component refers to a hydrophilic polymer of which the content is equal to or greater than 50% by mass with respect to the total mass of the hydrophilic processed portion. The content of the hydrophilic polymer is preferably equal to or greater than 70% by mass, and more preferably equal to or greater than 90% by mass.

(Antibacterial Agent)

Hereinafter, the antibacterial agent contained in the hydrophilic processed portion will be specifically described.

The hydrophilic processed portion contains at least a first antibacterial agent containing silver (hereinafter, simply referred to as “first antibacterial agent” as well) and a second antibacterial agent containing silver (hereinafter, simply referred to as “second antibacterial agent” as well) different from the first antibacterial agent. That is, the hydrophilic processed portion contains at least two kinds of antibacterial agents containing silver (hereinafter, simply referred to as “silver-based antibacterial agents” as well).

In the present embodiment, the hydrophilic processed portion may only contain at least one kind of silver as an antibacterial agent.

The type of the first antibacterial agent and the second antibacterial agent is not particularly limited as long as the antibacterial agents contain silver (silver atoms). Furthermore, the form of silver is not particularly limited, and for example, the silver is contained in the antibacterial agent in the form of metallic silver, a silver ion, a silver salt (including a silver complex), and the like. In the present specification, a silver complex included in the scope of a silver salt.

Examples of the silver salt include silver acetate, silver acetylacetonate, silver azide, silver acetylide, silver arsenate, silver benzoate, silver hydrogen fluoride, silver bromate, silver bromide, silver carbonate, silver chloride, silver chlorate, silver chromate, silver citrate, silver cyanate, silver cyanide, silver (cis,cis-1,5-cyclooctadiene)-1,1,1,5,5,5-hexafluoroacetylacetonate, silver diethyldithiocarbamate, silver(I) fluoride, silver(II) fluoride, silver 7,7-dimethyl-1,1,1,2,2,3,3-heptafluoro-4,6-octanedioate, silver hexafluoroantimonate, silver hexafluoroarsenate, silver hexafluorophosphate, silver iodate, silver iodide, silver isothiocyanate, potassium silver cyanide, silver lactate, silver molybdate, silver nitrate, silver nitrite, silver(I) oxide, silver(II) oxide, silver oxalate, silver perchlorate, silver perfluorobutyrate, silver perfluoropropionate, silver permanganate, silver perrhenate, silver phosphate, silver picrate monohydrate, silver propionate, silver selenate, silver selenide, silver selenite, silver sulfadiazine, silver sulfate, silver sulfide, silver sulfite, silver telluride, silver tetrafluoroborate, silver tetraiodocuriumate, silver tetratungstate, silver thiocyanate, silver p-toluenesulfonate, silver trifluoromethanesulfonate, silver trifluoroacetate, silver vanadate, and the like.

An example of the silver complex include a histidine-silver complex, a methionine-silver complex, a cysteine-silver complex, an aspartic acid-silver complex, a pyrrolidone carboxylic acid-silver complex, an oxotetrahydrofuran carboxylic acid-silver complex, an imidazole-silver complex, or the like.

Examples of the first antibacterial agent and the second antibacterial agent include organic antibacterial agents such as the aforementioned silver salts and inorganic antibacterial agents containing supports which will be described later, but the type of the antibacterial agents is not particularly limited.

Among the above antibacterial agents, in view of exhibiting an antibacterial action within a short period of time and/or being able to maintain the antibacterial agent properties for a long time (hereinafter, simply described as “in view of further improving the effects of the present invention” as well), the first antibacterial agent and the second antibacterial agent are preferably a silver-supporting support containing a support and silver supported on the support.

The type of the support is not particularly limited, and examples thereof include calcium zinc phosphate, calcium phosphate, zirconium phosphate, aluminum phosphate, calcium silicate, activated carbon, activated alumina, silica gel, zeolite, hydroxyapatite, titanium phosphate, potassium titanate, hydrous bismuth oxide, hydrous zirconium oxide, hydrotalcite, and the like. Examples of zeolite include natural zeolite such as chabazite, mordenite, erionite, and clinoptilolite, and synthetic zeolite such as type A zeolite, type X zeolite, and type Y zeolite.

The average particle diameter of the aforementioned silver-supporting support is not particularly limited. In view of further improving the effects of the present invention, the average particle diameter is preferably 0.1 to 10 μm, and more preferably 0.1 to 2 μm. The average particle diameter is a value obtained by measuring diameters of at least 10 random silver-supporting supports by using a microscope and calculating an arithmetic mean thereof.

As described above, silver may be contained in the antibacterial agent in any form such as a silver ion, metallic silver, and a silver salt.

As a suitable aspect of the second antibacterial agent, a silver-supporting support is preferable in which a porous substance (for example, zeolite, zirconium phosphate, aluminum phosphate, or calcium silicate) is used as a support.

Furthermore, as the most suitable aspect of the first antibacterial agent and the second antibacterial agent, in view of further improving the effects of the present invention, the first antibacterial agent preferably contains silver and one support selected from the group consisting of calcium zinc phosphate and calcium phosphate, and the second antibacterial agent preferably contains silver and a support formed of zeolite. In other words, the first antibacterial agent is a silver-supporting catalyst containing one support selected from the group consisting of calcium zinc phosphate and calcium phosphate and silver supported on the support, and the second antibacterial agent is a silver-supporting catalyst containing a support formed of zeolite and silver supported on the support.

The content of silver in the first antibacterial agent and the second antibacterial agent is not particularly limited. For example, in a case of the aforementioned silver-supporting support, the content of silver with respect to the total mass of the silver-supporting support is preferably 0.1% to 30% by mass, and more preferably 0.3% to 10% by mass.

The total content of the first antibacterial agent and the second antibacterial agent in the hydrophilic processed portion is not particularly limited. However, in view of further improving the effects of the present invention, it is preferable that the hydrophilic processed portion contains the first antibacterial agent and the second antibacterial agent such that the content of silver with respect to the total mass of the hydrophilic processed portion becomes 0.0001% to 1% by mass (preferably 0.001% to 0.1% by mass).

The amount of silver in the hydrophilic processed portion means the total amount of silver in the first antibacterial agent and silver in the second antibacterial agent.

The content of the first antibacterial agent (or the second antibacterial agent) in the hydrophilic processed portion is not particularly limited. However, in view of further improving the mechanical strength of the hydrophilic processed portion and further improving the effects of the present invention, the content of the first antibacterial agent with respect to the total mass of the hydrophilic processed portion is preferably 0.001% to 10% by mass, more preferably 0.01% to 5% by mass, and even more preferably 0.01% to 1% by mass.

In the present invention, the antibacterial agent may not be exposed on the surface of the hydrophilic processed portion.

The hydrophilic processed portion may contain other components in addition to the hydrophilic polymer and the antibacterial agent described above.

For example, the hydrophilic processed portion may contain a lubricant. In a case where the lubricant is contained in the hydrophilic processed portion, concavities and convexities can be formed on the outermost layer.

The average particle diameter of the lubricant is not particularly limited, but is preferably 0.5 μm to 30 μm, more preferably 0.5 μm to 20 μm, and even more preferably 6 μm to 10 μm.

The average particle diameter of the lubricant is obtained by measuring the particle diameters (diameters) of 100 random particles by using a microscope (for example, a scanning electron microscope) and calculating an arithmetic mean thereof. In a case where the shape of the lubricant is not a perfect circle, the major axis thereof is measured as the diameter.

The material of the lubricant is not particularly limited. Examples of the material include an inorganic compound (for example, a metal) and a resin, and among these, a resin is preferable. In a case where a resin is used, the amount of the radiation Ray absorbed is reduced. Therefore, a problem in that an artifact is superposed on a captured image or a problem in that the amount of radiation reaching the radiation detector 12 in the portable radiographic imaging device 10 is attenuated does not easily occur. As a result, it is possible to reduce the exposure dose for a patient as a subject.

(Characteristics of Hydrophilic Processed Portion)

The water contact angle of the surface of the hydrophilic processed portion is equal to or smaller than 30°. In view of further improving the antifogging properties and the removability of contaminants, the water contact angle is preferably equal to or smaller than 21°, and more preferably equal to or smaller than 15°. The lower limit thereof is not particularly limited. However, in view of the characteristics of materials used, the lower limit of the water contact angle is equal to or greater than 5° in many cases.

In a case where the water contact angle is greater than 30°, sufficient antibacterial properties or antifogging properties are not obtained, and the removability of contaminants becomes poor.

In the present specification, the water contact angle is measured based on a sessile drop method of JIS R 3257:1999. For measuring the water contact angle, LSE-ME1 (software 2win mini) manufactured by NiCK Corporation is used. More specifically, at room temperature (20° C.), 2 μl of droplets of pure water are dropped onto the surface of the hydrophilic processed portion which is kept horizontal, and a contact angle at a point in time when 20 seconds has elapsed from the dropping is measured.

The surface of the hydrophilic processed portion suitably has minute concavities and convexities. In a case where the surface has the minute concavities and convexities, the contact area between the hydrophilic processed portion and a subject can be reduced, and consequently, the amount of the contaminant derived from sebum and the like adhering to the hydrophilic processed portion can be reduced. Furthermore, the contact area between the hydrophilic processed portion and the contaminant can be reduced, and a void can be easily formed between the contaminant and the surface of the hydrophilized portion. Therefore, water or the like easily permeates the void, and as a result, the removability of the contaminant is improved.

In a case where the hydrophilic processed portion has the concavities and convexities particularly in a portion which comes into contact with a subject, the contact area between the hydrophilic processed portion and the skin of the subject such as a patient is reduced. Accordingly, the hydrophilic processed portion feels less sticky, and thus the discomfort the patient experiences at the time of imaging can be reduced.

The surface roughness Ra of the surface of the hydrophilic processed portion is not particularly limited, but is preferably 1 μm to 20 μm, more preferably 2 μm to 15 μm, and even more preferably 3 μm to 6 μm.

The surface roughness Ra is measured based on the method specified in JIS-B0601:2001. Specifically, by using a stylus scanning-type surface roughness tester, the surface roughness of 5 random sites on the surface of the hydrophilic processed portion is measured, and the average thereof is taken as the surface roughness Ra. It is also possible to measure the surface roughness Ra in the same manner as the stylus scanning-type method by using a laser microscope (for example, VK-X200 manufactured by KEYENCE CORPORATION) equipped with a “roughness meter mode”.

The average thickness of the hydrophilic processed portion is not particularly limited. In view of the removability of contaminants and the antibacterial properties, the average thickness is preferably 0.5 μm to 20 μm, and more preferably 1 μm to 10 μm.

The average thickness of the hydrophilic processed portion is measured by a method in which a sample piece is embedded in a resin, a cross section thereof is obtained by cutting with a microtome, and the obtained cross section is observed with a scanning electron microscope. The thicknesses at 10 random points in the hydrophilic processed portion are measured, and an arithmetic mean thereof is calculated.

(Substrate with Hydrophilic Processed Portion)

FIG. 4 is a schematic cross-sectional view of an example of a substrate with a hydrophilic processed portion of the present embodiment. As shown in FIG. 4, a substrate with a hydrophilic processed portion 40 has a substrate 42 and a hydrophilic processed portion 44 disposed on the substrate 42. The hydrophilic processed portion 44 may be disposed on at least a portion of the substrate 42.

(Characteristics of Substrate with Hydrophilic Processed Portion)

Regarding the substrate with a hydrophilic processed portion including the aforementioned substrate, the substrate constituting the portable radiographic imaging device 10, and a hydrophilic processed portion disposed on at least a portion of the surface of the substrate, provided that a silver content per unit area in the hydrophilic processed portion is P (ng/cm²) and a silver ion amount per unit area measured by an extraction test described below is Q (ng/cm²), P and Q satisfy relationships shown in Formula (1) and Formula (2) described below.

6.0≤P/Q Formula (1)

15.0≤Q Formula (2)

Hereinafter, first, the method for measuring P and Q will be specifically described.

The silver content P (ng/cm²) per unit area in the hydrophilic processed portion is a value obtained by dividing the amount of silver (ng) contained in the hydrophilic processed portion by the area (cm²) of the main surface of the hydrophilic processed portion. In FIG. 4, the area of the main surface of the hydrophilic processed portion means the area of a main surface 44a on the side opposite to the substrate 42 of the hydrophilic processed portion 44.

The higher the value of the silver content P, the greater the amount of silver contained in the hydrophilic processed portion. The magnitude of the silver content P is not particularly limited as long as the relationship shown in Formula (1) is satisfied. However, in view of further improving the effects of the present invention, the silver content P is preferably equal to or greater than 90 ng/cm², and more preferably equal to or greater than 120 ng/cm². The upper limit thereof is not particularly limited. However, from the viewpoint of preventing the discoloration or embrittlement of the film, the upper limit of the silver content P is preferably equal to or smaller than 10,000 ng/cm², and more preferably equal to or smaller than 1,000 ng/cm².

The silver content P is measured by a method in which the amount of silver in a hydrophilic processed portion is measured by atomic absorption spectrometry (contrAA 700 manufactured by Analytik Jena AG) by using a hydrophilic processed portion adjusted to have a predetermined size (area), and the silver content P is determined by dividing the obtained amount of silver by the aforementioned area. More specifically, all the silver contained in the hydrophilic processed portion is eluted, and the aforementioned atomic absorption spectrometry is performed using the obtained solution. Then, from a calibration curve plotted in advance, the amount of silver is determined and divided by the area of the hydrophilic processed portion, thereby obtaining the silver content P. As a method for eluting all the silver contained in the hydrophilic processed portion, a wet carbonization treatment is used which is generally known as a pre-treatment in inorganic analysis.

Hereinafter, a method of an extraction test will be specifically described.

In the extraction test, a 1/500 normal nutrient broth medium specified in JIS Z 2801:2010 is used as an extractant, and the temperature of the extractant is controlled within a range of 35±1° C. The extractant (amount: 9 mL) is brought into contact for 1 hour with the hydrophilic processed portion (area of the hydrophilic processed portion: 4 cm² (2 cm×2 cm)) in a substrate with a hydrophilic processed portion. As a method for bringing the hydrophilic processed portion into contact with the extractant, a method of immersing the substrate with a hydrophilic processed portion in the extractant is performed.

Then, after 1 hour, the substrate with a hydrophilic processed portion is recovered from the extractant, and the silver ion amount (ng) extracted into the extractant is measured. The silver ion amount in the extractant is measured using atomic absorption spectrometry (device name: contrAA 700 manufactured by Analytik Jena AG) and determined from a calibration curve that is plotted in advance.

At the time of measuring the silver ion amount, if necessary, it is preferable to add nitric acid (about 1 mL) to the extractant so as to improve the stability of measurement.

Thereafter, by dividing the obtained silver ion amount by a contact area (4 cm²) between the hydrophilic processed portion and the extractant, the silver ion amount Q per unit area (ng/cm²) is calculated. The contact area between the hydrophilic processed portion and the extractant means an area in which the hydrophilic processed portion and the extractant contact each other in a case where the hydrophilic processed portion is brought into contact with the extractant. For example, in FIG. 4, the contact area means the area of the main surface 44a that is on the side opposite to the substrate 42 side of the hydrophilic processed portion 44.

The silver ion amount Q obtained in this way shows a degree of elution (extraction) of silver ions from the hydrophilic processed portion, and satisfies the relationship of Formula (2). Formula (2) means that Q is equal to or greater than 15.0 ng/cm².

15.0≤Q Formula (2)

Particularly, in view of further improving the effects of the present invention, it is preferable that Q satisfies a relationship of Formula (3).

15.0≤Q≤25.0 Formula (3)

Furthermore, in view of further improving the effects of the present invention, Q is preferably 17.0 to 24.0 ng/cm², and more preferably 19.0 to 21.0 ng/cm².

In a case where the silver ion amount Q is less than 15.0 ng/cm², the antibacterial properties exhibited within a short period of time become poor.

The silver content P and the silver ion amount Q described above satisfy the relationship of Formula (1). Formula (1) means that P/Q is equal to or greater than 6.0. P/Q is a value obtained by dividing P by Q.

6.0≤P/Q Formula (1)

Particularly, in view of further improving the effects of the present invention, P/Q is preferably equal to or greater than 7.0, and more preferably equal to or greater than 10.0. The upper limit thereof is not particularly limited. However, because the antibacterial property-lasting effect is saturated, the upper limit of P/Q is preferably equal to or smaller than 20.0, and more preferably equal to or smaller than 15.0.

In a case where P/Q is less than 6, predetermined antibacterial properties cannot be exhibited over a long time.

The portable radiographic imaging device in which the substrate with a hydrophilic processed portion is disposed on the outer surface of the device has antibacterial properties that are exhibited within a short period of time and retained over a long time.

Particularly, in a case where the hydrophilic processed portion contains two kinds of silver-based antibacterial agents, the device has antibacterial properties retained over a long time. As a result of repeating a thorough study, the inventors of the invention of the present application obtained knowledge that in a case where two kinds of silver-based antibacterial agents are used by being mixed together, additivity is not established in the eluted Ag ion amount, but in a case where the above formulae are satisfied, excellent antibacterial properties can be retained over a long time.

(Method for Preparing Hydrophilic Processed Portion)

The method for preparing the aforementioned hydrophilic processed portion is not particularly limited, and known methods can be adopted. Examples of the method include a method of forming a hydrophilic processed portion through coating by using a composition containing the hydrophilic polymer and the antibacterial agent described above, a method of bonding a separately prepared polymer film containing a hydrophilic polymer and an antibacterial agent to a predetermined position, and the like.

Among these methods, in view of making it easier to adjust the thickness and surface asperity of the hydrophilic processed portion, the aforementioned method (coating method) is preferable in which a predetermined position is coated with a composition for forming a hydrophilic processed portion (hereinafter, simply referred to as “composition” as well) containing a monomer having a hydrophilic group and an antibacterial agent so as to form a coating film, and the coating film is subjected to a curing treatment.

The composition contains the monomer having a hydrophilic group and the antibacterial agent described above. Furthermore, the composition may contain other components (other monomers described above, a lubricant, and a solvent (water or an organic solvent)).

The composition may also contain a polymerization initiator. In a case where the composition contains a polymerization initiator, polymerization more efficiently proceeds in the coating film, and thus a hydrophilic processed portion having excellent mechanical strength is formed. The type of the polymerization initiator is not particularly limited, and an optimal type is selected according to the method of the curing treatment. For example, a thermal polymerization initiator or a photopolymerization initiator is selected. More specifically, examples of the polymerization initiator include aromatic ketones such as benzophenone and phenylphosphine oxide, α-hydroxyalkylphenone-based compounds (BASF IRGACURE 184, 127, 2959, DAROCUR 1173, and the like), phenylphosphine oxide-based compounds (MAPO: BASF LUCIRIN TPO and BAPO: BASF IRGACURE 819), and the like.

The content of the polymerization initiator contained in the composition is not particularly limited, but is preferably 0.1 to 15 parts by mass and more preferably 1 to 6 parts by mass, with respect to the total mass (100 parts by mass) of the monomer having a hydrophilic group and other monomers.

The coating method of the composition is not particularly limited, and known methods can be adopted.

Furthermore, the method of the curing treatment is not particularly limited, and examples thereof include a heating treatment or a light irradiation treatment.

The portable radiographic imaging device 10 according to the present embodiment is basically constituted as above. Hereinafter, a method will be described which is for forming the hydrophilic processed portion that is provided in at least a portion of the outer surface of the portable radiographic imaging device 10, that is, a predetermined site such as at least the irradiation surface 19, the LED lamp 35, the counting display portion 36, and the battery level display portion 37.

The portable radiographic imaging device 10 according to the present embodiment is provided with the hydrophilic processed portion at a predetermined site within the outer surface of the device. The method for providing the hydrophilic processed portion at a predetermined site within the outer surface is not particularly limited, and the hydrophilic processed portion may be provided by any method. For example, the hydrophilic processed portion may be provided by bonding a hydrophilic processed sheet having a hydrophilic processed portion to at least a portion of a predetermined site within the outer surface, that is, to all of the predetermined site or a portion of the predetermined site within the outer surface. Alternatively, the hydrophilic processed portion may be provided by forming a hydrophilic processed antibacterial film having a hydrophilic portion in at least a portion of a predetermined site of the outer surface, that is, in all of the predetermined site or a portion of the predetermined site within the outer surface.

(Hydrophilic Processed Sheet)

Next, the hydrophilic processed sheet will be described which is used for providing a hydrophilic processed portion at a predetermined site within the outer surface of the portable radiographic imaging device 10 of the present invention.

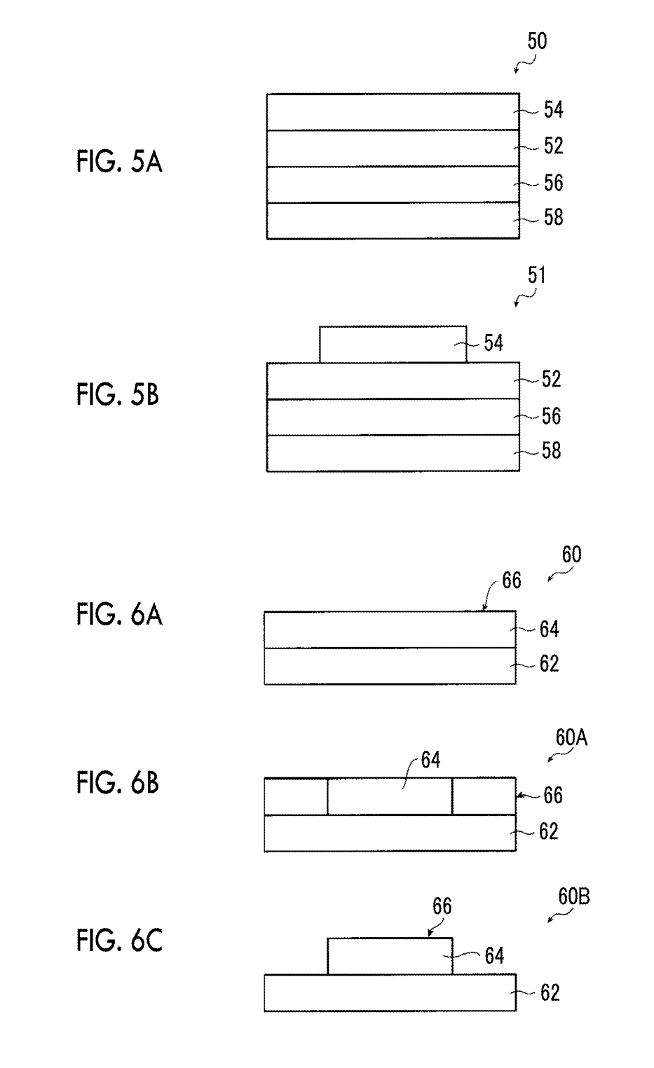

As shown in FIG. 5A, a hydrophilic processed sheet 50 of the present invention has a main sheet 52, a hydrophilic processed portion 54 which is formed on one lateral surface of the main sheet 52, a pressure sensitive adhesive layer 56 which is formed on the other surface of the main sheet 52 that is on the side opposite to one lateral surface, and a release sheet 58 which is laminated on a surface of the pressure sensitive adhesive layer 56 that is on the side opposite to the main sheet 52.

The hydrophilic processed sheet of the present invention is not limited to an aspect in which the hydrophilic processed portion 54 is formed on the entirety of one outer surface of the main sheet 52 as in the hydrophilic processed sheet 50 shown in FIG. 5A. The hydrophilic processed sheet of the present invention may be formed on a portion of one outer surface of the main sheet 52 as in a hydrophilic processed sheet 51 shown in FIG. 5B.

The hydrophilic processed sheets 50 and 51 of the present invention are used for forming a laminate of the hydrophilic processed portion 54 and the main sheet 52 on the outer surface of the aforementioned portable radiographic imaging device 10 of the present invention.

In the examples shown in FIGS. 5A and 5B, the hydrophilic processed sheets 50 and 51 have the pressure sensitive adhesive layer 56. Therefore, by peeling the release sheet 58 from the pressure sensitive adhesive layer 56 and bonding the pressure sensitive adhesive layer 56 to a predetermined site within the inner surface of the portable radiographic imaging device 10 that will become a surface for forming a hydrophilic processed portion, the laminate of the hydrophilic processed portion 54 and the main sheet 52 can be bonded to the surface for forming a hydrophilic processed portion by the pressure sensitive adhesive layer 56, and the hydrophilic processed portion 54 can be provided by being mounted on a predetermined site within the outer surface of the portable radiographic imaging device 10.

In the examples shown in FIGS. 5A and 5B, the hydrophilic processed sheets 50 and 51 have the pressure sensitive adhesive layer 56 in addition to the laminate of the hydrophilic processed portion 54 and the main sheet 52. However, the present invention is not limited thereto, and the hydrophilic processed sheets 50 and 51 may be constituted only with the laminate of the hydrophilic processed portion 54 and the main sheet 52. In a case where the hydrophilic processed sheets 50 and 51 are constituted only with the laminate of the hydrophilic processed portion 54 and the main sheet 52, by additionally forming an adhesive layer or the like by means of coating the surface for forming a hydrophilic processed portion or the surface of the main sheet 52 with an adhesive or the like, and bonding the laminate of the hydrophilic processed portion 54 and the main sheet 52 to the surface for forming a hydrophilic processed portion, the hydrophilic processed portion 54 can be formed.

Herein, because the hydrophilic processed portion 54 is the same as the aforementioned hydrophilic processed portion, the description thereof will not be repeated.

The main sheet 52 supports the hydrophilic processed portion 54 formed on the entirety or a partial region of one outer surface thereof. Although the hydrophilic processed portion 54 may be formed on the entirety or a portion of one outer surface of the main sheet 52, it is preferable that the hydrophilic processed portion 54 is formed on the entirety of one outer surface of the main sheet 52.

The main sheet 52 is not particularly limited as long as it can support the hydrophilic processed portion 54, and any material may be used. As the main sheet 52, a known sheet can be used. For example, it is possible to use a polyethylene terephthalate film (PET), a polybutylene terephthalate film (PBT), a polyimide film, a triacetyl cellulose film, and the like. As PET, for example, it is possible to use LUMIRROR U34 manufactured by TORAY INDUSTRIES, INC, COSMOSHINE A4300 manufactured by Toyobo Co., Ltd, O3916W manufactured by TEIJIN LIMITED, and the like. Furthermore, an easily adhesive layer may be provided on the surface thereof.

The thickness of the main sheet 52 is not particularly limited, and those having a thickness of 10 μm to 200 μm can be preferably used. In a case where the main sheet 52 is to be bonded to a resistive film-type touch panel, the main sheet 52 needs to conform to a flexible surface, and hence the thickness of the main sheet 52 is 10 μm to 100 μm and preferably 10 μm to 50 μm. In a case of a capacitance-type touch panel, in view of ease of bonding, it is possible to preferably use a main sheet 52 having a thickness of 50 μm to 100 μm.

The pressure sensitive adhesive layer 56 is used for bonding the laminate of the hydrophilic processed portion 54 and the main sheet 52 to the surface for forming a hydrophilic processed portion at a predetermined site within the outer surface of the portable radiographic imaging device 10 described above. The pressure sensitive adhesive layer 56 is not particularly limited as long as it enables the laminate of the hydrophilic processed portion 54 and the main sheet 52 to be bonded to the surface for forming a hydrophilic processed portion, and may be formed using a known pressure sensitive adhesive. The pressure sensitive adhesive usable in the pressure sensitive adhesive layer 56 is not particularly limited, and examples thereof include a (meth)acrylic pressure sensitive adhesive, a rubber-based pressure sensitive adhesive, a silicone-based pressure sensitive adhesive, a urethane-based pressure sensitive adhesive, a polyester-based pressure sensitive adhesive, and the like. In a case where the pressure sensitive adhesive layer is used for the surface of a touch panel, considering the facts that the pressure sensitive adhesive layer is repeatedly bonded and peeled and needs to be bonded while preventing air bubbles from entering, it is also possible to preferably use a self-adhesive pressure sensitive adhesive. Herein, the (meth)acrylic pressure sensitive adhesive refers to an acrylic pressure sensitive adhesive and/or a methacrylic pressure sensitive adhesive. As the (meth)acrylic pressure sensitive adhesive, it is possible to use a (meth)acrylic pressure sensitive adhesive used in a pressure sensitive sheet which will be described later.

The method for forming the pressure sensitive adhesive layer is not particularly limited, and examples thereof include a coating method, a printing method, a bonding method, and the like. Among these, it is possible to preferably use a method of installing the pressure sensitive adhesive layer by coating and a method of forming the pressure sensitive adhesive layer by bonding a pressure sensitive sheet, and the method of forming the pressure sensitive adhesive layer by bonding a pressure sensitive sheet is more preferable.

The thickness of the pressure sensitive adhesive layer 56 is not particularly limited, but is preferably 1 μm to 30 μm. In a case where the thickness of the pressure sensitive adhesive layer is equal to or greater than 1 μm, the film can be stably formed by co-extrusion. In a case where the thickness is equal to or smaller than 30 μm, the material costs are reduced. At this time, for enhancing the adhesion, it is preferable to increase the thickness of the pressure sensitive adhesive layer in consideration of the viscosity thereof, because a contact area between the pressure sensitive adhesive layer and an object coated with the pressure sensitive adhesive layer is easily increased by increasing the thickness of the pressure sensitive adhesive layer. The thickness of the pressure sensitive adhesive layer is preferably 2 μm to 20 μm, and more preferably 3 μm to 15 μm.

The adhesion of the pressure sensitive adhesive layer 56 is not particularly limited, and is preferably within a range of 2 cN/25 mm to 20 cN/25 mm for use. In a case where the adhesion is equal to or greater than 2 cN/25 mm, when the pressure sensitive adhesive layer is used by being bonded to the surface of a touch panel or the like, the pressure sensitive adhesive layer is not easily detached. In a case where the adhesion is equal to or smaller than 20 cN/25 mm, the film can be smoothly peeled at the time of peeling.

The release sheet 58 remains bonded to the pressure sensitive adhesive layer 56 until the hydrophilic processed sheet 50 is used so as to protect the pressure sensitive adhesive layer 56. The release sheet 58 is not particularly limited as long as it can protect the pressure sensitive adhesive layer 56, and a known release sheet 58 can be used. For example, it is possible to use a release agent such as a silicone-based compound, a long-chain alkyl-based compound, or polyvinyl alcohol-carbamate.

The thickness of the release sheet 58 is not particularly limited, but is preferably 1 μm to 30 μm. In a case where the thickness of the release sheet is equal to or greater than 1 μm, the film can be stably formed by co-extrusion. In a case where the thickness is equal to or smaller than 30 μm, the material costs are reduced. The thickness of the release sheet is preferably 2 μm to 20 μm, and more preferably 3 μm to 15 μm.

(Hydrophilic Processed Antibacterial Film)

Next, the hydrophilic processed antibacterial film will be described which is formed directly at a predetermined site within the outer surface of the portable radiographic imaging device such that a hydrophilic processed portion is provided at a predetermined site within the outer surface of the portable radiographic imaging device of the present invention.

FIGS. 6A, 6B, and 6C show a base with a hydrophilic processed antibacterial film (hereinafter, simply referred to as base with an antibacterial film) in which a hydrophilic processed antibacterial film is formed directly on a base constituting a predetermined site of the housing of the portable radiographic imaging device.

First, a base with an antibacterial film 60 shown in FIG. 6A has a substrate 62 and a hydrophilic portion 64 which is formed on one lateral surface (upper surface in the example shown in the drawing) of the substrate 62. The hydrophilic portion 64 constitutes a hydrophilic processed antibacterial film 66 of the present invention.

The hydrophilic processed antibacterial film 66 of the present invention is not limited to what is shown in FIG. 6A that is constituted with the hydrophilic portion 64 formed on the entirety of one outer surface of the substrate 62. Similarly to a substrate with an antibacterial film 60A shown in FIG. 6B, the hydrophilic processed antibacterial film 66 may be formed on the entirety of one outer surface of the substrate 62 so as to include the hydrophilic portion 64 formed on a portion of one outer surface of the substrate 62. Alternatively, similarly to a substrate with an antibacterial film 60B shown in FIG. 6C, the hydrophilic processed antibacterial film 66 may be constituted with the hydrophilic portion 64 formed on a portion of one outer surface of the substrate 62.

The substrate 62 in the substrates with an antibacterial film 60, 60A, and 60B shown in FIGS. 6A, 6B, and 6C is a member constituting the surface for forming a hydrophilic processed portion of a predetermined site of the portable radiographic imaging device 10. For example, in the portable radiographic imaging device 10 shown in FIGS. 1 to 3, the substrate 62 corresponds to at least the irradiation surface 19, the LED lamp 35, the counting display portion 36, and the battery level display portion 37.

The hydrophilic portion 64 of the substrates with an antibacterial film 60, 60A, and 60B shown in FIGS. 6A, 6B, and 6C is disposed on at least a portion of the surface of the substrate 62. Specifically, the hydrophilic portion 64 may be disposed on the entirety of one surface of the substrate 62 similarly to the substrate with an antibacterial film 60 shown in FIG. 6A, or may be disposed only on a portion of the surface of the substrate 62 similarly to the substrates with an antibacterial film 60A and 60B shown in FIGS. 6A and 6C. Furthermore, the substrate 62 may constitute the entirety of the hydrophilic processed antibacterial film 66 similarly to the substrates with an antibacterial film 60 and 60B shown in FIGS. 6A and 6C, or may constitute only a portion of the hydrophilic processed antibacterial film 66 similarly to the substrate with an antibacterial film 60A shown in FIG. 6B.

Examples of the hydrophilic portion 64 include the hydrophilic processed portion of the present invention, specifically, the hydrophilic processed portion forming a predetermined site constituting at least a portion of the outer surface of the aforementioned portable radiographic imaging device 10, such as a hydrophilic portion having exactly the same constitution as the hydrophilic processed portion 54 of the hydrophilic processed sheets 50 and 51 shown in FIGS. 5A and 5B.

That is, because the hydrophilic portion of the present invention has exactly the same constitution as the hydrophilic processed portion of the present invention described above, the description of the hydrophilic portion will not be repeated.

The hydrophilic processed antibacterial film 66 of the substrates with an antibacterial film 60, 60A, and 60B shown in FIGS. 6A, 6B, and 6C is a film which is disposed on at least a portion of the surface of the substrate 62 and shows an antibacterial action. At least a portion of the hydrophilic processed antibacterial film 66 is hydrophilic. That is, at least a portion of the hydrophilic processed antibacterial film 66 is constituted with the hydrophilic portion 64.

Specifically, the hydrophilic processed antibacterial film 66 may be disposed on the entirety of one surface of the substrate 62 similarly to the substrates with an antibacterial film 60 and 60A shown in FIGS. 6A and 6B, or may be disposed only on a portion of the surface of the substrate 62 similarly to the substrate with an antibacterial film 60B shown in FIG. 6C. Furthermore, the entirety of the hydrophilic processed antibacterial film 66 may be constituted with the hydrophilic portion 64 similarly to the substrates with an antibacterial film 60 and 60B shown in FIGS. 6A and 6C. Alternatively, only a portion of the hydrophilic processed antibacterial film 66 may be constituted with the hydrophilic portion 64 similarly to the substrate with an antibacterial film 60A shown in FIG. 6B.

The hydrophilic processed antibacterial film contains an antibacterial agent contained in the aforementioned hydrophilic processed portion. The antibacterial agent and the antibacterial agent containing silver (silver-based antibacterial agent) used herein are the same as the antibacterial agent and the silver-based antibacterial agent described above. Therefore, the description thereof will not be repeated.

In the present embodiment, the content of the antibacterial agent or the silver-based antibacterial agent in the hydrophilic processed antibacterial film is not particularly limited as in the case of the hydrophilic processed portion. However, in view of further improving the effects of the present embodiment, the hydrophilic processed antibacterial film preferably contains the antibacterial agent or the silver-based antibacterial agent such that the content of the antibacterial agent (or silver) with respect to the total mass of the hydrophilic processed antibacterial film becomes 0.001 to 20 wt % (preferably 0.001 to 5 wt %).

The portable radiographic imaging device 10 according to the present embodiment is basically constituted as above. Next, the operation and effect thereof will be described.

In order to obtain a radiograph of a subject, first, at least the irradiation surface 19 of the portable radiographic imaging device 10 and suitably the entirety of the outer surface of the housing 18 is cleaned. That is, the housing 18 is wiped with a wiper containing a disinfectant solution. As the disinfectant solution, an aqueous ethanol solution or an aqueous sodium hypochlorite solution is preferably used.

As described above, the outer surface of the housing 18 of the portable radiographic imaging device 10 is provided with a hydrophilic processed portion. Furthermore, in the hydrophilic processed portion, a water contact angle is equal to or smaller than 30° even in a dark place not irradiated with light. Therefore, even in a case where the portable radiographic imaging device 10 is stored in a dark place, the outer surface of the housing 18 exhibits sufficient antifogging properties and hydrophilicity.

Due to the hydrophilicity, the outer surface of the housing 18 is sufficiently wetted with the disinfectant solution. In other words, the disinfectant solution sufficiently wets and spreads over the outer surface of the housing 18. Accordingly, even in a case where bacteria remain on the outer surface of the housing 18 at this point in time, the disinfectant solution comes into contact with the bacteria for a long period of time. Furthermore, because the hydrophilic processed portion on the outer surface of the housing 18 contains an antibacterial agent as described above, the antibacterial agent acts on the bacteria. Consequently, a bactericidal ability can be further improved compared to the related art, and the bacterial multiplication can be inhibited.

Due to the sufficient antifogging properties, it is possible to prevent the outer surface of the LED lamp 35, the counting display portion 36, and the battery level display portion 37 from becoming foggy and to prevent the deterioration of the visibility.

That is, even in a case where the portable radiographic imaging device 10 is stored in a dark place, the device can be sterilized immediately after being taken out of the dark place.

Furthermore, because the hydrophilic processed portion contains the antibacterial agent as described above, the portable radiographic imaging device in which the substrate with the hydrophilic processed portion is disposed on the outer surface of the device has antibacterial properties that are exhibited within a short time and last over a long time.

In addition, due to the sufficient antifogging properties of the hydrophilic processed portion, in the portable radiographic imaging device in which the substrate with the hydrophilic processed portion is disposed on the outer surface of the device, it is possible to prevent a wrong panel from being mounted and to easily check the center position of a panel at the time of imaging. Furthermore, the battery level or the number of sheets of images captured can be easily checked without a mistake.

In a state where the irradiation surface 19 of the housing 18 of the portable radiographic imaging device 10 cleaned as above is coming into contact with a subject, a physician or a radiological technician (radiographer) irradiates an imaging site of the subject with the radiation Ray from a radiation source. The radiation Ray is transmitted through the imaging site of the subject, passes through the irradiation surface 19 of the portable radiographic imaging device 10, and reaches the scintillator 21 of the radiation detector 12.

The scintillator 21 emits fluorescence (visible light) in an amount according to the transmission amount of the radiation Ray. Meanwhile, in the sensor portions provided in the TFT board 20, a charge in an amount according to the amount (emission amount) of the fluorescence is generated and accumulated. The information on the charge is read out by the control portion, and as a result, a radiograph of the imaging site of the subject is obtained.

The portable radiographic imaging device 10 is used in an operating room, an emergency room, and the like in some cases. In these cases, the blood or body fluid of a patient (subject) is likely to adhere to the housing 18. In order to remove this type of contaminant, a method of washing the housing 18 with running water is considered. However, in a case where such a method is used, a battery mounting portion or a connector connecting portion may be wetted with water, and thus the device may break down.

Therefore, after imaging ends, the housing 18 is wiped with a wiper containing a disinfectant solution such as an aqueous ethanol solution or an aqueous sodium hypochlorite solution. In this case, because the outer surface of the housing 18 is also provided with the hydrophilic processed portion as described above, the disinfectant solution wets and spreads on the outer surface of the housing 18, and the outer surface is sufficiently wetted with the disinfectant solution.

Accordingly, in a case where a contaminant adheres to the housing 18, water or the disinfectant solution goes in between the hydrophilic processed portion and the contaminant. As a result, the contaminant is easily detached from the housing 18. That is, the contaminant can be easily removed.

Furthermore, because the disinfectant solution stays on the outer surface of the housing 18 for a long time, even though the bacteria from the contaminant remain on the outer surface, the disinfectant solution comes into contact with the bacteria for a long time. In addition, because the hydrophilic processed portion on the outer surface of the housing 18 contains an antibacterial agent, the antibacterial agent acts on the bacteria. Accordingly, a bactericidal ability can be further improved compared to the related art, and the bacterial multiplication can be inhibited.

As described above, in a case where the outer surface of the housing 18 is provided with the hydrophilic processed portion, it is possible to remove the contaminant from the portable radiographic imaging device, which has a difficulty of being cleaned with running water in the related art, and to easily perform sterilization with an improved bactericidal ability in a case where sterilization is performed using a disinfectant solution. Furthermore, because the hydrophilic processed portion on the outer surface of the housing 18 contains the antibacterial agent, it is possible to inhibit the bacteria from multiplying after the device is sterilized with the disinfectant solution. Accordingly, even though the contaminant remains on the device, the bacterial multiplication can be inhibited by the action of the antibacterial agent.

According to the present embodiment, the housing 18 of the portable radiographic imaging device 10 can be easily cleaned after imaging. Furthermore, because the hydrophilic processed portion on the outer surface of the housing 18 contains an antibacterial agent, it is possible to obtain advantages that bacterial multiplication is inhibited, and the housing 18 remains clean for a long time.

### Embodiment 2

The hydrophilic processed portion according to Embodiment 1 contains at least the first antibacterial agent containing silver and the second antibacterial agent containing silver which is different from the first antibacterial agent. A hydrophilic processed portion according to Embodiment 2 is not particularly limited as long as it contains at least one kind of silver as an antibacterial agent. That is, Embodiment 1 and Embodiment 2 have the same constitution and the same operation, except that different antibacterial agents are used.

(Antibacterial Agent)

In Embodiment 2, the type of an antibacterial agent containing silver (hereinafter, referred to as a silver-based antibacterial agent as well) is not particularly limited, as long as the antibacterial agent contains silver (silver atoms). The aspect of silver used in Embodiment 2 and specific examples thereof are the same as in Embodiment 1.

Examples of the silver-based antibacterial agent include organic antibacterial agents such as a silver salt (silver complex) and inorganic antibacterial agents containing a support, but the type of the antibacterial agent is not particularly limited.

Among the silver-based antibacterial agents, in view of further improving the light fastness of the hydrophilic processed portion and/or in view of further improving the antibacterial properties (hereinafter, simply described as “in view of further improving the effects of Embodiment 2 as well”), a silver-supporting support containing a support and silver supported on the support is preferable.

The type of the support used in Embodiment 2 is the same as in Embodiment 1. However, in view of further improving the effects of the Embodiment 2, ceramics are preferable as the support.

The average particle diameter of the silver-supporting support used in Embodiment 2 is the same as in Embodiment 1.

The content of silver in the silver-based antibacterial agent is not particularly limited. For example, in a case of the aforementioned silver-supporting support, the content of silver with respect to the total mass of the silver-supporting support is preferably 0.1% to 10% by mass, and more preferably 0.3% to 5% by mass.

The content of the silver-based antibacterial agent in the hydrophilic processed portion is not particularly limited. However, in view of further improving the effects of Embodiment 2, it is preferable that the hydrophilic processed portion contains the silver-based antibacterial agent such that the content of silver with respect to the total mass of the hydrophilic processed portion becomes 0.001% to 20% by mass (preferably 0.001% to 5% by mass).

In a case where an organic antibacterial agent is used as the silver-based antibacterial agent, the content of the antibacterial agent is not particularly limited. However, in view of further improving the mechanical strength of the hydrophilic processed portion and further improving the effects of Embodiment 2, the content of the antibacterial agent is preferably 1% to 4% by mass with respect to the total mass of the hydrophilic processed portion.

In a case where an inorganic antibacterial agent is used as the silver-based antibacterial agent, the content of the antibacterial agent is not particularly limited. However, in view of further improving the mechanical strength of the hydrophilic processed portion and further improving the effects of Embodiment 2, the content of the antibacterial agent with respect to the total mass of the hydrophilic processed portion is preferably 0.001% to 10% by mass, and more preferably 0.01% to 5% by mass.

(Characteristics of Hydrophilic Processed Portion)

The water contact angle of the surface of the hydrophilic processed portion is equal to or smaller than 30°. In view of further improving the antifogging properties and the removability of contaminants, the water contact angle is preferably equal to or smaller than 21°, and more preferably equal to or smaller than 15°. The lower limit thereof is not particularly limited. However, in view of the characteristics of materials used, the lower limit of the water contact angle is equal to or greater than 5° in many cases.

In a case where the water contact angle is greater than 30°, sufficient antibacterial properties or antifogging properties are not obtained, and the removability of contaminants becomes poor.

In the present specification, the water contact angle is measured based on a sessile drop method of JIS R 3257:1999. For measuring the water contact angle, LSE-ME1 (software 2win mini) manufactured by NiCK Corporation is used. More specifically, at room temperature (20° C.), 2 μl of droplets of pure water are dropped onto the surface of the hydrophilic processed portion which is kept horizontal, and a contact angle at a point in time when 20 seconds has elapsed from the dropping is measured.

The average thickness of the hydrophilic processed portion is not particularly limited. However, in view of the removability of contaminants and the antibacterial properties, the average thickness is preferably 0.5 μm to 20 μm, and more preferably 1 μm to 10 μm.

The average thickness of the hydrophilic processed portion is measured by a method in which a sample piece is embedded in a resin, a cross section thereof is obtained by cutting with a microtome, and the obtained cross section is observed with a scanning electron microscope. The thicknesses at 10 random points in the hydrophilic processed portion are measured, and an arithmetic mean thereof is calculated.

(Method for Preparing Hydrophilic Processed Portion)

The method for preparing the hydrophilic processed portion containing the aforementioned antibacterial agent is the same as in Embodiment 1.

(Substrate with Hydrophilic Processed Portion)

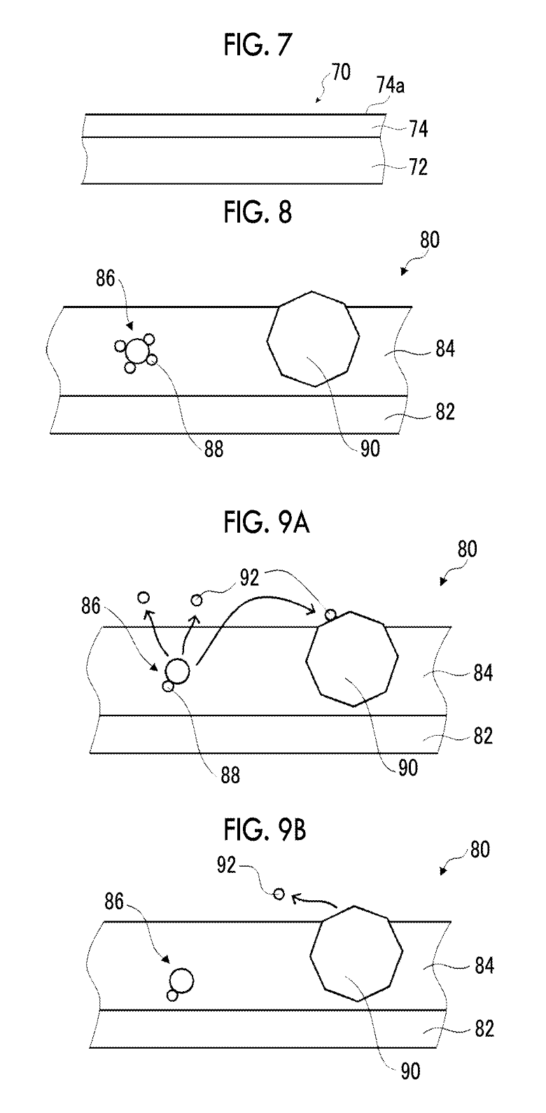

FIG. 7 shows a substrate with a hydrophilic processed portion according to Embodiment 2. A substrate with a hydrophilic processed portion 70 has a substrate 72 and a hydrophilic processed portion 74 disposed on the substrate 72. The portable radiographic imaging device of the present embodiment has the substrate with a hydrophilic processed portion 70 on the outer surface of the device. The hydrophilic processed portion 74 may be disposed on at least a portion of the surface of the substrate 72.

(Characteristics of Substrate with Hydrophilic Processed Portion)

In the substrate with a hydrophilic processed portion 70 containing the aforementioned antibacterial agent, a silver ion amount per unit area that is measured by an extraction test which will be described later is 15 to 50 ng/cm². In view of further improving the effects of Embodiment 2, the silver ion amount is preferably 15 to 40 ng/cm², and more preferably 15 to 30 ng/cm².

In a case where the silver ion amount is less than 15 ng/cm², the antibacterial properties become poor. In a case where the silver ion amount is greater than 50 ng/cm², the light fastness becomes poor. That is, the base with a hydrophilic processed portion (portable radiographic imaging device) has excellent light fastness and antibacterial properties exhibited within a short time.

The method of the extraction test used in Embodiment 2 is the same as in Embodiment 1.

The hydrophilic processed portion containing the aforementioned antibacterial agent has sufficient antifogging properties and excellent antibacterial properties showing an antibacterial action within a short time. Furthermore, the hydrophilic processed portion has the effect of light fastness by which the hydrophilic processed portion is not discolored even being exposed to external light for a long time.

Due to the sufficient antifogging properties of the hydrophilic processed portion, in the portable radiographic imaging device in which the substrate with a hydrophilic processed portion is disposed on the outer surface of the device, it is possible to prevent a wrong panel from being mounted and to easily check the center position of a panel at the time of imaging. Furthermore, it is possible to easily check the battery level or the number of sheets of images captured without a mistake.

In addition, the substrate with a hydrophilic processed portion according to Embodiment 2 has effects that makes it possible to obtain sufficient antifogging properties, sufficient visibility, sufficient light fastness, and antibacterial properties exhibited within a short time.

The substrate 72 and the hydrophilic processed portion 74 of the substrate with a hydrophilic processed portion 70 constitute the main sheet 52 and the hydrophilic processed portion 54 of the hydrophilic processed sheet 50 according to Embodiment 1 and the substrate 62 and constitute the hydrophilic portion 64 of the base with a hydrophilic processed antibacterial film (base with an antibacterial film) 60, respectively.

### Third Embodiment

FIG. 8 shows a substrate with a hydrophilic processed portion according to a third embodiment. A substrate with a hydrophilic processed portion 80 has a substrate 82 and a hydrophilic processed portion 84 disposed on the substrate 82. The portable radiographic imaging device 10 of the present embodiment has the substrate with a hydrophilic processed portion 80 on at least a portion of the outer surface of the device. The hydrophilic processed portion 84 contains an antibacterial agent 86 containing silver 88 and a porous support 90 which can adsorb silver ions.

In Embodiment 1 and Embodiment 2, the hydrophilic processed portion contains a hydrophilic polymer and an antibacterial agent. However, the hydrophilic processed portion according to Embodiment 3 contains a hydrophilic polymer, an antibacterial agent containing silver, and a porous support which can adsorb silver ions.

In the substrate including the hydrophilic processed portion according to Embodiment 3, an average particle diameter Da (μm) of the antibacterial agent, an average particle diameter Db (μm) of the porous support, and an average thickness T (μm) of hydrophilic processed portion satisfy the relationships of Formula (4) and Formula (5).

T/Da>3.0 Formula (4)

T/Db≤3.0 Formula (5)

As shown in FIG. 8, the antibacterial agent 86 satisfying the condition of Formula (4) is buried in the hydrophilic processed portion 84 in many cases, and the porous support 90 satisfying the condition of Formula (5) is easily exposed on the surface of the hydrophilic processed portion 84.

Embodiment 1 and Embodiment 3 have the same constitution and the same operation, except that different hydrophilic processed portions are adopted.

Hereinafter, the present embodiment will be specifically described.

(Hydrophilic Polymer)

The hydrophilic polymer is the same as in Embodiment 1.

(Antibacterial Agent Containing Silver)

The type of the antibacterial agent containing silver (hereinafter, referred to as a silver-based antibacterial agent as well) is not particularly limited as long as the antibacterial agent contains silver (silver atoms). The aspect of silver used in Embodiment 3 and specific examples thereof are the same as Embodiment 1.

Examples of the silver-based antibacterial agent include organic antibacterial agents such as a silver salt and inorganic antibacterial agents containing a support, but the type thereof is not particularly limited.

Among these, in view of exhibiting antibacterial properties within a shorter time and/or in view of being able to retain antibacterial properties for a longer period of time (hereinafter, simply described as “in view of further improving the effects of Embodiment 3” as well), the antibacterial agent is preferably a silver-supporting support containing a support and silver supported on the support. The type of the support used in Embodiment 3 is the same as in Embodiment 1.

The average particle diameter of the antibacterial agent (preferably the aforementioned silver-supporting support) is not particularly limited as long as the condition of Formula (4) is satisfied. In view of further improving the effects of Embodiment 3, the average particle diameter is preferably 0.1 to 10 μm, more preferably equal to or greater than 0.1 μm and less than 2.0 μm, and even more preferably 0.3 to 1.0 μm.

Herein, by using a laser diffraction/scattering-type particle size distribution measurement apparatus manufactured by HORIBA, Ltd, a 50% by volume cumulative diameter (D50) is measured three times, and the average of the values obtained by the measurement performed three times is used as the aforementioned average particle diameter.

In Embodiment 3, as one of the suitable aspects of the silver-based antibacterial agent, in view of further improving the effects of the present invention, an antibacterial agent can be exemplified which contains silver and at least one support selected from the group consisting of calcium zinc phosphate and calcium phosphate. In other words, the antibacterial agent is preferably a silver-supporting catalyst containing one support selected from the group consisting of calcium zinc phosphate and calcium phosphate and silver supported on the support.

(Porous Support which can Adsorb Silver Ions)

The porous support which can adsorb silver ions refers to a support having a number of pores that can adsorb silver ions. The porous support is not particularly limited regarding the diameter or shape of pores, the volume of pores, the density of pores, the specific surface area, and the like. Specifically, it is possible to arbitrarily use an inorganic porous support such as activated carbon, zeolite, activated carbon fiber, silica gel, activated clay, alumina, or diatom earth or an organic polymer porous support such as pulp, fiber, paper, cloth, non-woven cloth, wood, or wood powder. Examples of zeolite include natural zeolite such as chabazite, mordenite, erionite, and clinoptilolite, and synthetic zeolite such as type A zeolite, type X zeolite, and type Y zeolite.

The porous support may support silver. That is, a silver-supporting porous substance (corresponding to a so-called antibacterial agent) containing a porous support and silver supported on the porous support may be used. In a case where silver is supported on the porous support (that is, in a case of the silver-supporting porous substance), the type of the porous support supporting silver is different from the type of the aforementioned antibacterial agent.

As described above, the hydrophilic processed portion includes one kind of porous support (porous substance) selected from the group consisting of a porous support which can adsorb silver ions and a porous support which supports silver and can adsorb silver ions.

The silver supported on the porous support may be contained in any form such as a silver ion, metallic silver, and a silver salt.

Particularly, in view of further improving the effects of Embodiment 3, the hydrophilic processed portion preferably includes a porous support supporting silver. In other words, the hydrophilic processed portion preferably includes a silver-supporting porous substance containing silver and a porous support. Examples of the porous support preferably include zeolite. That is, the porous support is preferably a silver-supporting catalyst containing a support formed of zeolite and silver supported on the support.

The average particle diameter of the porous support which can adsorb silver ions is not particularly limited as long as the condition of Formula (5) described above is satisfied. However, in view of further improving the effects of the present invention, the particle diameter of the porous support is preferably 0.1 to 20 μm, more preferably 1.0 to 10 μm, and even more preferably 2.0 to 5.0 μm.

Herein, by using a laser diffraction/scattering-type particle size distribution measurement apparatus manufactured by HORIBA, Ltd, a 50% by volume cumulative diameter (D50) is measured three times, and the average of the values obtained by the measurement performed three times is used as the aforementioned average particle diameter.

(Characteristics of Hydrophilic Processed Portion and Method for Preparing Hydrophilic Processed Portion)

The characteristics of the hydrophilic processed portion containing the antibacterial agent and the porous support described above and the method for preparing the hydrophilic processed portion are the same as in Embodiment 1.

(Characteristics of Substrate with Hydrophilic Processed Portion)

As described above and as shown in FIG. 8, in the substrate (portable radiographic imaging device) including the hydrophilic processed portion having the aforementioned antibacterial agent, the average particle diameter Da (μm) of the antibacterial agent, the average particle diameter Db (μm) of the porous support, and the average thickness T (μm) of the hydrophilic processed portion satisfy the relationships of Formula (4) and Formula (5).

T/Da>3.0 Formula (4)

T/Db≤3.0 Formula (5)

The substrate with the hydrophilic processed portion has antibacterial properties which are exhibited within a short time and last over a long period of time (long time).

Formula (4) means that a ratio (T/Da) between the average thickness T of the hydrophilic processed portion and the average particle diameter Da of the antibacterial agent is higher than 3.0. Particularly, in view of further improving the effects of Embodiment 3, the ratio (T/Da) is preferably equal to or higher than 3.1, and more preferably equal to or higher than 3.2. The upper limit thereof is not particularly limited, but is generally equal to or lower than 10 in many cases. In view of further improving the effects of Embodiment 3, the upper limit of the ratio (T/Da) is preferably equal to or lower than 6.0. In a case where the relationship of Formula (4) is satisfied, the antibacterial agent can be easily buried in the hydrophilic processed portion, and hence an excess of silver ions can be inhibited from being eluted from the hydrophilic processed portion.

Formula (5) means that a ratio (T/Db) between the average thickness T of the hydrophilic processed portion and the average particle diameter Db of the porous support is equal to or lower than 3.0. Particularly, in view of further improving the effects of Embodiment 3, the ratio (T/Db) is preferably equal to or lower than 2.5, and more preferably equal to or lower than 2.0. The lower limit thereof is not particularly limited. However, in view of further improving the flatness of the hydrophilic processed portion, the lower limit of the ratio (T/Db) is preferably equal to or higher than 1.0. In a case where the relationship of Formula (5) is satisfied, the porous support more easily protrudes from the surface of the hydrophilic processed portion, and hence silver ions are easily adsorbed onto the support.

The relationship between the average particle diameter Da of the antibacterial agent and the average particle diameter Db of the porous support is not particularly limited. However, in view of further improving the effects of Embodiment 3, a ratio (Db/Da) between the average particle diameter Da and the average particle diameter Db is preferably equal to or lower than 4.5, and it is preferable that the ratio (Db/Da) satisfies the relationship of Formula (6) shown below.

Db/Da≤3.5 Formula (6)

The lower limit of Db/Da is not particularly limited. However, in view of further improving the effects of Embodiment 3, the lower limit of Db/Da is preferably equal to or higher than 1.0, and more preferably equal to or higher than 2.0.

The content of silver in the antibacterial agent is not particularly limited. For example, in a case where the antibacterial agent is a silver-supporting support, the content of silver with respect to the total mass of the silver-supporting support is preferably 0.1% to 30% by mass, and more preferably 0.3% to 10% by mass.

In a case where silver is supported on the porous support, the amount of silver supported is not particularly limited. However, the amount of silver supported is preferably 0.1% to 30% by mass and more preferably 0.3% to 10% by mass with respect to the total mass of silver and the porous support.

The total content of the antibacterial agent and the porous support in the hydrophilic processed portion is not particularly limited. However, in view of further improving the effects of Embodiment 3, it is preferable that the hydrophilic processed portion contains the antibacterial agent and the porous support such that the content of silver with respect to the total mass of the hydrophilic processed portion becomes 0.0001% to 1% by mass and preferably becomes 0.001% to 0.1% by mass.

The amount of silver in the hydrophilic processed portion means the total amount of the silver in the antibacterial agent and the silver supported on the porous support.

The content of the antibacterial agent in the hydrophilic processed portion is not particularly limited. However, in view of further improving the mechanical strength of the hydrophilic processed portion and further improving the effects of Embodiment 3, the content of the antibacterial agent with respect to the total mass of the hydrophilic processed portion is preferably 0.001% to 10% by mass, more preferably 0.01% to 5% by mass, even more preferably 0.01% to 2.5% by mass, and particularly preferably greater than 1.0% by mass and equal to or smaller than 2.5% by mass.

The content of the porous support in the hydrophilic processed portion is not particularly limited. However, in view of further improving the effects of Embodiment 3, the content of the porous support with respect to the total mass of the hydrophilic processed portion is preferably equal to or smaller than 10% by mass, more preferably equal to or smaller than 5% by mass, even more preferably equal to or smaller than 0.8% by mass, and particularly preferably equal to or smaller than 0.5% by mass. The lower limit thereof is not particularly limited, but is preferably equal to or greater than 0.001% by mass and more preferably equal to or greater than 0.01% by mass.

Next, with reference to FIGS. 9A and 9B, a mechanism will be described by which the substrate with a hydrophilic processed portion 80 according to Embodiment 3 exhibits antibacterial properties.

First, as shown in FIG. 9A, the silver 88 in the antibacterial agent 86 is ionized, and silver ions 92 are eluted from the hydrophilic processed portion 84 and act on the bacteria and the like on the hydrophilic processed portion 84. At this time, some of the eluted silver ions 92 are adsorbed onto the surface of the porous support 90 without acting on the bacteria. In a case where the silver ions 92 are continuously eluted from the antibacterial agent 86, the amount of silver in the antibacterial agent 86 is reduced, and hence the amount of the silver eluted is reduced. Then, the silver ions 92 adsorbed onto the porous support 90 start to be eluted to the outside as shown in FIG. 9B such that the equilibrium relationship of silver ions is maintained, and in this way, the antibacterial properties are retained. That is, in a case where the porous support 90 is included in the hydrophilic processed portion 84, the silver ions 92 eluted from the antibacterial agent 86 can be temporarily held, and the silver ions 92 can be eluted again after the passage of a predetermined time (after the amount of silver ions eluted from the antibacterial agent 86 is reduced). Accordingly, the antibacterial properties can be maintained over a longer period of time than in a case where the porous support 90 is not included in the hydrophilic processed portion 84.

The substrate with a hydrophilic processed portion according to Embodiment 3 (portable radiographic imaging device) has excellent antifogging properties and antibacterial properties which are exhibited within a short time and retained over a long period of time (long time).

The substrate 82 and the hydrophilic processed portion 84 of the substrate with a hydrophilic processed portion 80 constitute the main sheet 52 and the hydrophilic processed portion 54 of the hydrophilic processed sheet 50 according to Embodiment 1 and constitute the substrate 62 and the hydrophilic portion 64 of the base with a hydrophilic processed antibacterial film (base with antibacterial film) 60 respectively.

### Embodiment 4

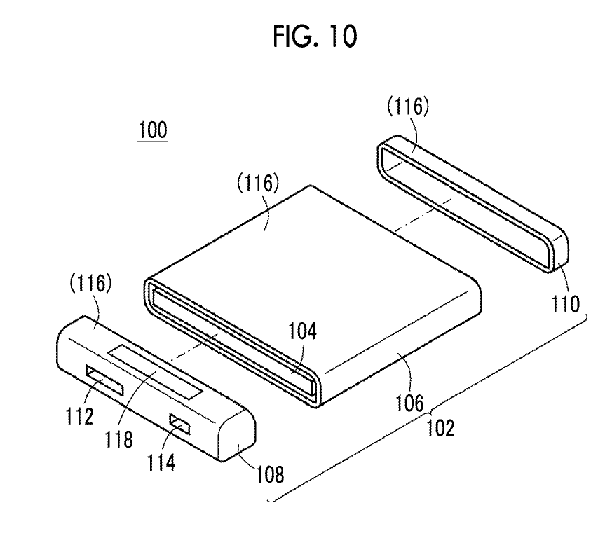

FIG. 10 is a schematic perspective view showing the entirety of a portable radiographic imaging device (electronic cassette-type imaging device) 100 according to Embodiment 4. The portable radiographic imaging device 100 is constituted with a housing 102 and a radiation detector 104 accommodated in the housing.

The radiation detector 104 includes a scintillator, sensor portions, and the like not shown in the drawing. Furthermore, the radiation detector 104 is provided with a charge amplifier IC, a communication portion (none of these are shown in the drawing), and the like.

The housing 102 includes a body member 106 of which both ends in the longitudinal direction are open ends and a first cap member 108 and a second cap member 110 that close the open ends. The body member 106 has a cavity on the inside thereof and is in the form of a so-called cylinder. All of the body member 106, the first cap member 108, and the second cap member 110 may be constituted with a resin material that can transmit radiation.

The first cap member 108 is provided with a battery mounting portion 112 and a connector connecting portion 114. From a battery (not shown in the drawing) mounted on the battery mounting portion 112, a driving current is supplied, and through a connector (not shown in the drawing) mounted on the connector connecting portion 114, wire communication is performed between the portable radiographic imaging device 100 and external instruments. It goes without saying that wireless communication may be performed instead of the wire communication.

Furthermore, as shown in FIG. 10, the first cap member 108 may be provided with a display portion 118 or the like. The display portion 118 is constituted with an LED lamp or the like, and is used for displaying the driving state and the like of the portable radiographic imaging device 100.

One end face of the body member 106, the first cap member 108, and the second cap member 110 constituting the housing 102 forms an irradiation surface 116 irradiated with radiation and comes into contact with a subject (not shown in the drawing) as a patient. At least the portion of the body member 106, the first cap member 108, and the second cap member 110 that comes into contact with the subject (that is, the surface irradiated with radiation) and the top of the display portion 118 are provided with a hydrophilic processed portion similarly to the aforementioned portable radiographic imaging device 10 according to Embodiment 1. The hydrophilic processed portion may be provided not only in the portion of the body member 106, the first cap member 108, and the second cap member 110 that comes into contact with the subject but also on the entirety of the outer surface that can come into contact with a radiographer.

In this case, because the open ends of the body member 106 accommodating the radiation detector 104 on the inside thereof are closed by the first cap member 108 and the second cap member 110, the housing 102 in the form of a so-called monocoque is formed, and the portable radiographic imaging device 100 is constituted. The body member 106 only needs to be able to accommodate the radiation detector 104 on the inside thereof, and both ends thereof do not have to be open ends. For example, only one end thereof may be an open end, and the open end may be closed by a cap member.

In the portable radiographic imaging device 100, the same effect as in the portable radiographic imaging device 10 according to Embodiment 1 is obtained. Herein, in a case where a portion coming into contact with a subject is very far away from an external instrument-connecting portion such as the connector connecting portion 114, for example, in the case of a radiographic imaging device in which the external instrument-connecting portion is not to be wetted with water even though the portion coming into contact with a subject is washed with water, the portion coming into contact with a subject may be washed with flowing water. In this case, due to the presence of the hydrophilic processed portion, water also permeates the space between a contaminant and the outer surface of the device. Accordingly, it is possible to easily remove the contaminant and to inhibit the multiplication of bacteria.

Furthermore, because the outer surface of the display portion 118 has antifogging properties, excellent visibility is also obtained. Therefore, it is possible to accurately ascertain the driving state of the portable radiographic imaging device 100 and the like.

In the portable radiographic imaging device 100 shown in FIG. 10, the second cap member 110 can be mounted on the body member 106 through fitting, adhesion, or welding. In a case where a structure is adopted in which the first cap member 108 is detachably mounted through adhesion or welding, the portion other than the first cap member 108 can be immersed in a washing solution. At this time, it is preferable to cap the battery mounting portion 112 and the connector connecting portion 114 with an elastomer so as to prevent these portions from being accidentally wetted with water, although the elastomer is not shown in the drawing. Furthermore, it is preferable to make the first cap member 108 by using an O-ring or the like in advance, although the O-ring is not shown in the drawing.

Even in this case, because the housing 102 is also provided with the hydrophilic processed portion, it is possible to easily remove the contaminant and to inhibit the multiplication of bacteria.

In the present embodiment, the first cap member 108 may be provided with a detachable handle or a storage handle, although such a handle is not shown in the drawing. Furthermore, a self-pressure sensitive adhesive sheet may be bonded to the housing 102 having a monocoque structure similarly to the portable radiographic imaging device 100 shown in FIG. 10, because such a sheet is easily bonded to the housing 102.

### Embodiment 5

(Mammography Device)

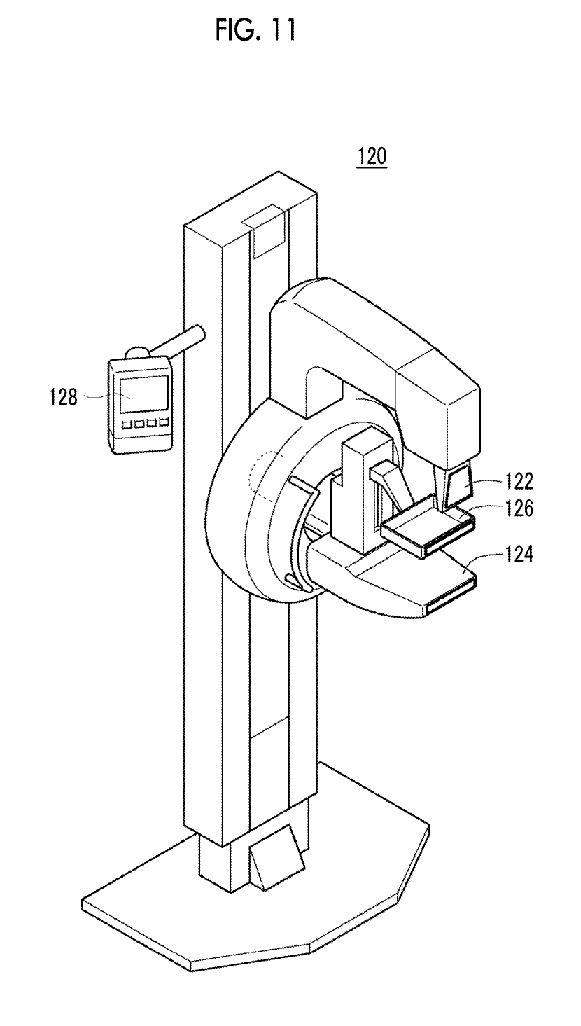

The radiographic imaging device of the present invention may be a mammography device 120 shown in FIG. 11. In this case, similarly to the portable radiographic imaging device 10 according to Embodiment 1, a hydrophilic processed portion, which contains an antibacterial agent and has undergone antibacterial processing, can be suitably provided in a face guard 122, a breast support 124, or a breast compression plate 126 that is a portion coming into contact with a subject. Furthermore, the hydrophilic processed portion having undergone antibacterial processing can also be suitably provided in a display portion 128 showing the driving state and the like of the mammography device 120.

The lipstick or sebum of a patient adheres to the face guard 122 in some cases. Moreover, oozing breast milk, blood resulting from bleeding at the time of biopsy (mammotome biopsy), or sebum adheres to the breast support 124 and the breast compression plate 126 in some cases. In a case where the hydrophilic processed portion is provided in the face guard 122, the breast support 124, and the breast compression plate 126, it is possible to remove the lipstick, breast milk, blood, and sebum simply by wiping, and to improve the bactericidal ability compared to the related art because the hydrophilic processed portion exhibits excellent wettability with respect to the disinfectant solution at the time of sterilization.

In addition, in a case where the hydrophilic processed portion is provided on the surface of the display portion 128, even though the indoor humidity is high, the antifogging properties of the display portion 128 can be maintained.

Furthermore, in a case where the hydrophilic processed portion having undergone antibacterial processing is provided, it is possible to inhibit the multiplication of bacteria remaining after wiping or to kill the bacteria. It is also possible to suitably use a method of providing minute concavities and convexities on the surface of the substrate of the hydrophilic processed portion and a method of adding a lubricant to the hydrophilic processed portion.

### Embodiment 6

(Radiographic Imaging Device for Upright Radiography)

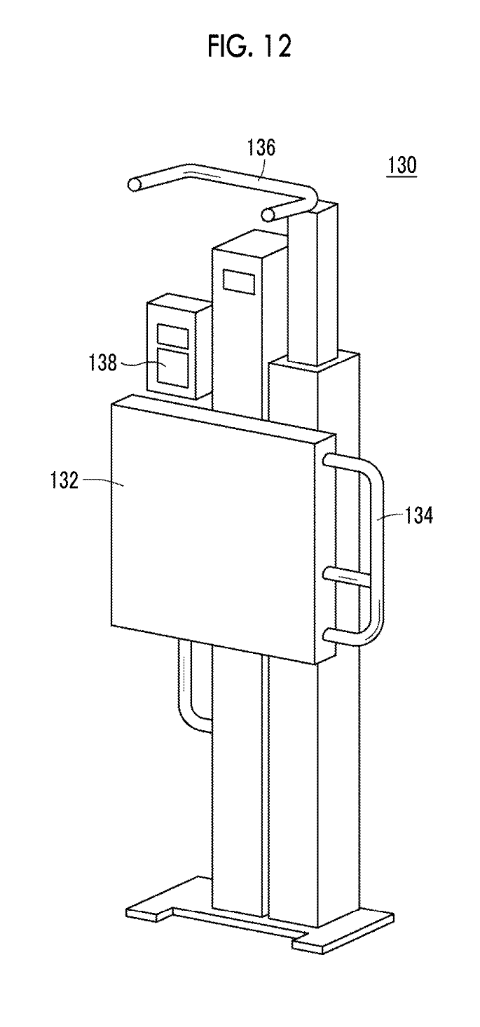

The radiographic imaging device of the present invention may be a radiographic diagnostic device 130 for upright radiography shown in FIG. 12. In this case, similarly to the portable radiographic imaging device 10 according to Embodiment 1, an imaging board 132 coming into contact with a subject at the time of imaging or grips 134 and 136 a subject grips at the time of imaging are suitably provided with a hydrophilic processed portion which contains an antibacterial agent and has undergone antibacterial processing. Furthermore, the hydrophilic processed portion having undergone antibacterial processing can be suitably provided in other portions including an operation panel portion 138 touched by a radiographer.

In addition, in a case where a hydrophilic processed portion also having excellent antifogging properties is provided on the surface of the operation panel portion 138, the visibility of the operation panel portion 138 can be maintained even though the indoor humidity is increased.

(CR Cassette)

The outer surface of a CR cassette, which accommodates an imaging plate used for computed radiography (CR) at the time of imaging, can be suitably provided with the same hydrophilic processed portion as in Embodiment 1, that is, the hydrophilic processed portion containing an antibacterial agent and having undergone antibacterial processing. In this case, similarly to the portable radiographic imaging device according to Embodiment 1, the surface of the imaging surface coming into contact with a subject or the entirety of the outer surface of the CR cassette that can be touched by a radiographer is suitably provided with such a hydrophilic processed portion.

(Grid)

In addition, the surface of a grid, which is used for removing scattering radiation and improving contrast at the time of imaging performed using a portable radiographic imaging device or a CR cassette, is suitably provided with the same hydrophilic processed portion as in Embodiment 1, that is, the hydrophilic processed portion containing an antibacterial agent and having undergone antibacterial processing.

The present invention is not limited to the aforementioned examples, and the outer surface of a radiographic imaging device which needs antifogging properties and comes into contact with a subject and to which an infectious contaminant can adhere can be provided with the same hydrophilic processed portion as in Embodiment 1, that is, the hydrophilic processed portion containing an antibacterial agent and having undergone antibacterial processing.

### Embodiment 7

(Medical Mask)

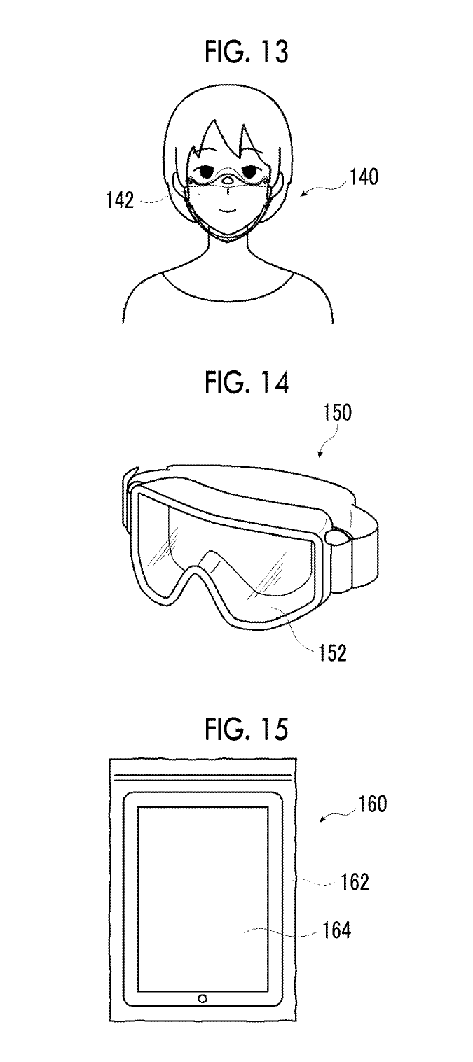

The medical instrument of the present invention may be a medical mask 140 shown in FIG. 13.

The medical mask 140 is a transparent mask formed of plastic. The hydrophilic processed portion of the present invention is provided on the inner surface 142 of the mask. It is suitable for the hydrophilic processed portion to be provided for the mouth of patients or healthcare professionals.

In a case where the hydrophilic processed portion of the present invention having antibacterial properties and antifogging properties is provided on the inner surface of the mask, it is possible to check the mouth without taking off the mask. Furthermore, the mask is not easily contaminated with lipstick or the like, and even in a case where the mask is stained with lipstick or the like, the lipstick stain can be removed simply by wiping. In addition, the mask exhibits excellent wettability with respect to a disinfectant solution at the time of sterilization, the bactericidal ability can be further improved compared to the related art.

### Embodiment 8

(Medical Goggle)

The medical instrument of the present invention may be a medical goggle 150 shown in FIG. 14.

In a case where the hydrophilic processed portion of the present invention having antibacterial properties or antifogging properties is provided on an outer surface 152 of the medical goggle, excellent visibility can be secured even in a cold environment. Furthermore, in a case where the hydrophilic processed portion is provided on the inner surface of the medical goggle, the goggle is not easily contaminated with sweat or sebum, and even though the goggle is contaminated with sweat or sebum, the sweat or sebum can be removed simply by wiping. In addition, the goggle exhibits excellent wettability with respect to a disinfectant solution at the time of sterilization, and the bactericidal ability can be further improved compared to the related art.

### Embodiment 9

(Transparent Sterile Medical Bag)

The medical instrument of the present invention may be a transparent sterile medical bag 160 shown in FIG. 15.

In a case where an instrument such as a tablet PC 164 or the like that is unsterilizable is brought into an operating room, sometimes the PC is used in a state of being packed in a bag. However, in this case, the inside of the bag becomes foggy, and sometimes the visibility deteriorates. At this time, in a case where the hydrophilic processed portion of the present invention having undergone antibacterial processing is provided on an inner surface 162 of the sterile bag, the antifogging properties can be maintained.

The present invention is not limited to the aforementioned examples, and the same hydrophilic processed portion as in Embodiment 1, that is, the hydrophilic processed portion containing an antibacterial agent and having undergone antibacterial processing can be provided at sites which need antifogging properties and come into contact with a subject and to which infectious contaminants can adhere, for example, the inner surface of a showcase for food, the inner surface of food packaging bag, the inner wall of a bathroom, and the like.

In a case where the hydrophilic processed portion of the present invention having excellent antifogging properties or antibacterial properties is provided on the inner surface of a showcase for food or a food packaging bag, the food disposed in the showcase for food or in the food packaging bag can be easily seen, and the food does not get spoiled due to the water droplets that adhere to the inside of the showcase or the food packaging bag and fall onto the food.

### EXAMPLES

### Examples 1 to 5 and Comparative Examples 1 to 3

(Preparation of Curable Composition)

By mixing the following components together, a curable composition was prepared.

| | |
|---|---|
| Hydrophilic monomer: Miramer M4004 (manufactured | 76 parts by mass |
| by Toyo Chemicals Co., Ltd.) | |
| Cross-linking agent: A-DPH (manufactured by | 21 parts by mass |
| SHIN-NAKAMURA CHEMICAL CO., LTD.) | |
| Polymerization initiator: IRGACURE (manufactured | 3 parts by mass |
| by BASF SE) | |
| Solvent component (1): methyl alcohol | 15 parts by mass |
| Solvent component (2): propylene glycol | 35 parts by mass |
| monomethyl ether | |

(Preparation of Antibacterial Agent)

(First Antibacterial Agent: Antibacterial Agent Containing Silver Supported on Calcium Zinc Phosphate)

By reacting calcium hydroxide with zinc oxide and phosphoric acid, a phosphate was obtained. Silver nitrate was added to the phosphate, and the mixture was washed, filtered, dried, and ground, thereby obtaining a first antibacterial agent (silver-supporting support). The average particle diameter of the obtained first antibacterial agent was 1 μm, and the silver ion content in the antibacterial agent was equivalent to 3% by mass.

(Second Antibacterial Agent: Antibacterial Agent Containing Silver Supported on Zeolite)

Zeolite was brought into contact with an aqueous silver nitrate solution, such that ion-exchangeable ions in the zeolite were substituted with silver ions, thereby obtaining a second antibacterial agent. The average particle diameter of the obtained second antibacterial agent was 1.51 μm, and the silver ion content in the antibacterial agent was equivalent to 0.5% by mass.

### Examples and Comparative Examples

The first antibacterial agent and the second antibacterial agent were added to and mixed with the curable composition prepared as above, according to the ratio (part by mass) shown in Table 1 with respect to 100 parts by mass of the total solid contents in the composition, thereby preparing a curable composition for forming a hydrophilic processed portion.

<Various Evaluations>

(1) Antibacterial Properties

A polycarbonate sheet CARBOGLASS (CFR110C manufactured by ASAHI GLASS CO., LTD) was coated with the obtained curable composition for forming a hydrophilic processed portion. The composition was dried for 30 minutes at 60° C., and then the monomer was cured by UV irradiation. In this way, a hydrophilic processed portion was formed, and an evaluation sample (substrate with a hydrophilic processed portion) was prepared.

By using the obtained evaluation samples of the examples and the comparative examples, the following evaluation was performed. The results are summarized in Table 1.

(Measurement of Silver Content P)

The evaluation samples prepared in the examples and the comparative examples were cut such that the hydrophilic processed portion had a size (area) of 25 cm². Then, the obtained samples were subjected to a wet carbonization treatment, and the silver content (ng) was measured by atomic absorption spectrometry (contrAA 700 manufactured by Analytik Jena AG) with using a calibration curve plotted in advance. The obtained value was divided by the area of the hydrophilic processed portion, thereby determining the silver content P (ng/cm²).

(Measurement of Silver Ion Amount Q)

The evaluation samples prepared in the examples and the comparative examples were cut such that the hydrophilic processed portion had a size (area) of 4 cm². The cut evaluation samples were immersed in 9 ml of a bacterial liquid (1/500 normal nutrient broth medium specified in JIS Z 2801:2010) for 1 hour at 35° C. Then, 1 ml of nitric acid was added to the bacterial liquid, and the silver ion amount was measured by atomic absorption spectrometry (contrAA 700 manufactured by Analytik Jena AG) by using a calibration curve plotted in advance. The obtained value was divided by the area of the hydrophilic processed portion, thereby determining the silver ion amount Q (ng/cm²).

(Evaluation of initial antibacterial properties)

For evaluating the antibacterial properties, based on the evaluation method described in JIS Z 2801:2010, a test was performed by changing the contact time with the bacterial liquid to 1 hour from 24 hours. The bacterial count (bacteria/cm²) was measured after each test, and the initial antibacterial properties were evaluated according to the standards shown below. As bacterial species, E. coli was used. In order to check the validity of the test, a polycarbonate sheet (CARBOGLASS CFR110C manufactured by ASAHI GLASS CO., LTD.) not provided with a hydrophilic processed portion was also evaluated. As a result, it was confirmed that the bacterial count (bacteria/cm²) after an hour of contact was equal to or greater than 6.2×10² (bacteria/cm²). For practical use, “A” or “B” is preferable.

“A”: the bacterial count is less than 1/cm².

“B”: the bacterial count is equal to or greater than 1/cm² and less than 10/cm². “C”: the bacterial count is equal to or greater than 10/cm².

(Evaluation of Antibacterial Properties after Durability Test)

In order to evaluate whether the evaluation sample prepared according to the aforementioned procedure retains antibacterial properties over a long time, the following durability test was performed. Generally, in a case where contaminants adhere to the hydrophilic processed portion disposed on the surface of a member, the hydrophilic processed portion is wiped with a damp cloth in many cases. At this time, the elution of silver into water occurs and becomes the main cause of the deterioration of antibacterial properties. Accordingly, in a case where excellent antibacterial properties are exhibited even after the following abrasion test, it is understood that the antibacterial properties can be retained over a long period of time even after the hydrophilic processed portion is used.

During the durability test, the surface of the hydrophilic processed portion of the evaluation sample (substrate with a hydrophilic processed portion) prepared according to the aforementioned procedure was rubbed 36,000 times with a damp cloth under a load of 500 g. Then, the test for evaluating antibacterial properties was performed by the method described above, and the antibacterial properties were evaluated according to the following standards. As bacterial species, E. coli was used. As the damp cloth, a polyester cloth (trade name: ANTICON) immersed in pure water was used. For practical use, “A” or “B” is preferable.

“A”: the bacterial count was less than 1/cm². “B”: the bacterial count was equal to or greater than 1/cm² and less than 10/cm². “C”: the bacterial count was equal to or greater than 10/cm².

“Silver content rate (wt %)” in Table 1 means the content (% by mass) of silver contained in the hydrophilic processed portion with respect to the total mass of the hydrophilic processed portion.

(2) Antifogging Properties

The outer surface of the counting display portion 36 and the battery level display portion 37 of the portable radiographic imaging device 10 shown in FIG. 3 was coated with the obtained curable composition for forming a hydrophilic processed portion, followed by curing (ultraviolet irradiation treatment), thereby providing a hydrophilic processed portion containing an antibacterial agent and having undergone antibacterial processing. The average thickness of the hydrophilic processed portion was about 2 μm. The water contact angle of the hydrophilic processed portion provided on the portable radiographic imaging device obtained in the examples and the comparative examples was measured by the aforementioned method.

(Evaluation of Antifogging Properties)

For evaluating antifogging properties, the portable radiographic imaging devices 10 obtained in Examples 1 to 5 and Comparative Examples 1 to 3 were kept in an environment with a temperature of 35° C. and a humidity of 95% for 1 hour, and the visibility of the hydrophilic processed portion of each of the portable radiographic imaging devices 10 was subjected to sensory evaluation by being visually observed by five researchers.

In the sensory evaluation of visibility, in a case where the moisture occurring on the surface of the portable radiographic imaging device 10 was scattered, and no fogging was observed within the hydrophilic processed portion, the device was graded A. In a case where although slight fogging was observed, the visibility of the numbers or the like displayed on the counting display portion 36 and the battery level display portion 37 were not affected at all, the device was graded B. In a case where although mild fogging was observed, the visibility of the numbers or the like displayed on the counting display portion 36 and the battery level display portion 37 were not affected, and the numbers or the like could be sufficiently read, the device was graded C. In a case where fogging occurred, and it was difficult to read the numbers or the like displayed on the counting display portion 36 and the battery level display portion 37, the device was graded D. In a case where dew condensation was observed in addition to fogging, the visibility was extremely poor, and the numbers or the like displayed on the counting display portion 36 and the battery level display portion 37 could not be read, the device was graded E.

The results are summarized in Table 1.

TABLE 1

| | Hydrophilic processed portion | | | | | | | Evaluation | | |
|---|---|---|---|---|---|---|---|---|---|---|
| | Content of first | Content of second | | | | Silver | Water | Evaluation | Evaluation | Evaluation of |
| | antibacterial | antibacterial | Silver content | Silver ion | | content | contact | of | of initial | antibacterial |
| | agent | agent | P | amount Q | | rate | angle | antifogging | antibacterial | properties after |
| | (part by mass) | (part by mass) | (ng/cm²) | (ng/cm²) | P/Q | (wt %) | (°) | properties | properties | durability test |
| Comparative | 0.5 | 0.5 | 70 | 12.3 | 5.7 | 0.018 | 10 | A | B | C |
| Example 1 | | | | | | | | | | |
| Example 1 | 1.0 | 0.5 | 130 | 15.7 | 8.3 | 0.033 | 10 | A | A | B |
| Example 2 | 1.5 | 0.5 | 190 | 18.6 | 10.2 | 0.048 | 10 | A | A | A |
| Example 3 | 2.0 | 0.5 | 250 | 21.1 | 11.8 | 0.063 | 10 | A | A | A |
| Example 4 | 2.5 | 0.5 | 310 | 23.7 | 13.1 | 0.078 | 10 | A | A | A |
| Comparative | 1.0 | 0.05 | 121 | 24 | 5.0 | 0.030 | 10 | A | A | C |
| Example 2 | | | | | | | | | | |
| Example 5 | 1.0 | 0.15 | 123 | 20 | 6.2 | 0.031 | 10 | A | A | B |
| Comparative | 0.5 | 0 | 60 | 14.1 | 4.3 | 0.015 | 10 | A | B | C |
| Example 3 | | | | | | | | | | |

As shown in Table 1, it was confirmed that the substrate with a hydrophilic processed portion of the present invention has excellent antifogging properties and excellent antibacterial properties that are exhibited within a short time and last over a long time. Particularly, it was confirmed that in a case where P/Q is equal to or higher than 10 as shown in Examples 2 to 4, better effects can be obtained.

In contrast, in Comparative Examples 1 to 3 not satisfying predetermined conditions, the effects of exhibiting excellent antifogging properties were obtained, but regarding the antibacterial properties that last over a long time, excellent effects were not obtained.

The above results clearly show the effects of Embodiment 1 of the present invention.

### Examples 6 to 10 and Comparative Examples 4 to 6

(Preparation of Antibacterial Agent)

A silver ceramic particle dispersion liquid (manufactured by Fuji Chemical Industries, Ltd., average particle diameter: 0.8 μm) was added to and mixed with cured compositions prepared by the same method as in Examples 1 to 5 and Comparative Examples 1 to 3 described above according to the ratio shown in “Mass of antibacterial agent” in Table 2, thereby preparing curable compositions for forming a hydrophilic processed portion.

<Evaluation>

(1) Evaluation of Antibacterial Properties and Light Fastness

A polycarbonate sheet (CARBOGLASS CFR110C manufactured by ASAHI GLASS CO., LTD.) was coated with the obtained curable composition for forming a hydrophilic processed portion such that a hydrophilic processed portion having a thickness shown in Table 2 was obtained. The composition was dried for 30 minutes at 60° C., and the monomer was cured by UV irradiation. In this way, a hydrophilic processed portion was formed, and an evaluation sample (substrate with a hydrophilic processed portion) was prepared.

In Comparative Example 6, a mass ratio between the hydrophilic monomer and a cross-linking agent was controlled such that a predetermined water contact angle was obtained.

(Method for Evaluating Antibacterial Properties)

For evaluating the antibacterial properties, based on the evaluation method described in JIS Z 2801:2010, a test was performed by changing the contact time with the bacterial liquid to 1 hour from 24 hours. The bacterial count (bacteria/cm²) was measured after each test, and the antibacterial properties were evaluated according to the standards shown below. As bacterial species, E. coli was used. For practical use, “A” or “B” is preferable. The results are shown in Table 2.

“A”: the bacterial count was less than 5/cm².

“B”: the bacterial count was equal to or greater than 5/cm² and less than 10/cm².

“C”: the bacterial count was equal to or greater than 10/cm².

(Light Fastness Test)

By using a xenon weatherometer (manufactured by Suga Test Instruments Co., Ltd.), the evaluation sample was irradiated for 10 hours at a power of 60 W/m², and then the discoloration of the hydrophilic processed portion in the sample was visually evaluated from the viewpoints shown below. For practical use, “A” is preferable. The results are shown in Table 2.

“A”: no discoloration occurred.

“B”: discoloration occurred.

By using the evaluation sample obtained in each of the examples and the comparative examples, the aforementioned extraction test was performed.

Furthermore, the water contact angle of the hydrophilic processed portion in the evaluation sample obtained in each of the examples and the comparative examples was measured by the aforementioned method. The results are shown in Table 2.

In Table 2, “Mass of antibacterial agent (% by mass)” means the content of the antibacterial agent (% by mass) with respect to the mass of the components of the cured substance (total mass of solid contents in the cured composition) in the hydrophilic processed portion.

In Table 2, the column of “Ag ion amount” means the silver ion amount per unit area measured by the extraction test.

(2) Evaluation of Antifogging Properties

The outer surface of the counting display portion 36 and the battery level display portion 37 of the portable radiographic imaging device 10 was coated with the obtained curable composition for forming a hydrophilic processed portion, followed by curing (ultraviolet irradiation treatment), thereby providing a hydrophilic processed portion containing an antibacterial agent and having undergone antibacterial processing. The average thickness of the hydrophilic processed portion was about 2 μM.

The water contact angle of the hydrophilic processed portion provided on the outer surface of the portable radiographic imaging device obtained in the examples and the comparative examples was measured by the aforementioned method.

The evaluation method of the antifogging properties was the same as the evaluation method used in Examples 1 to 5 and Comparative Examples 1 to 3 described above.

The results are summarized in Table 2.

TABLE 2

| | Hydrophilic processed portion | | | | | | |
|---|---|---|---|---|---|---|---|
| | Mass of | | | Evaluation | | | |
| | antibacterial | Film | | | | | Water |
| | agent | thickness | Ag ion amount | Antibacterial | Light | Antifogging | contact angle |
| | (% by mass) | (μm) | (ng/cm²) | properties | fastness | properties | (°) |
| Example 6 | 0.5 | 4.0 | 15.8 | A | A | B | 25 |
| Example 7 | 0.7 | 4.0 | 19.1 | A | A | B | 20 |
| Example 8 | 1.0 | 4.0 | 23.2 | A | A | B | 22 |
| Example 9 | 1.5 | 4.0 | 25.0 | A | A | B | 23 |
| Example 10 | 2.0 | 5.0 | 40.0 | A | A | B | 25 |
| Comparative | 0.5 | 2.0 | 6.3 | C | A | B | 23 |
| Example 4 | | | | | | | |
| Comparative | 3.0 | 5.0 | 63.2 | A | B | B | 21 |
| Example 5 | | | | | | | |
| Comparative | 1.5 | 4.0 | 20.2 | B | A | C | 50 |
| Example 6 | | | | | | | |

As shown in Table 2, the substrate with a hydrophilic processed portion of the present invention had excellent antifogging properties, excellent antibacterial properties that were exhibited within a short time, and excellent light fastness.

In contrast, in Comparative Example 4 in which the silver ion amount was smaller than a predetermined range, excellent antibacterial properties were not obtained. In Comparative Example 5 in which the silver ion amount was greater than a predetermined range, the light fastness was poor. In Comparative Example 6 in which the water contact angle was larger than a predetermined range, excellent antifogging properties were not obtained, and the antibacterial properties were poor.

The above results clearly show the effects of Embodiment 2 of the present invention.

### Examples 11 to 28 and Comparative Examples 7 and 8

(Preparation of Antibacterial Agent)

### Synthesis Example 1: Antibacterial Agent Containing Silver Supported on Calcium Zinc Phosphate

By reacting calcium hydroxide with zinc oxide and phosphoric acid, a phosphate was obtained. Silver nitrate was added to the phosphate, and the mixture was washed, filtered, dried, and ground, thereby obtaining an antibacterial agent A. The average particle diameter of the obtained antibacterial agent A was 0.9 μm, and the silver ion content in the antibacterial agent A with respect to the total mass of the antibacterial agent was equivalent to 3% by mass.

### Synthesis Example 2: Antibacterial Agent Containing Silver Supported on Calcium Zinc Phosphate

An antibacterial agent B was obtained according to the same procedure as in Synthesis Example 1, except that grinding was performed such that the average particle diameter became 0.5 μm after the grinding. The silver ion content in the antibacterial agent B with respect to the total mass of the antibacterial agent was equivalent to 1% by mass.

### Synthesis Example 3: Porous Support Supporting Silver

Zeolite was brought into contact with an aqueous silver nitrate solution such that ion-exchangeable ions in the zeolite were substituted with silver ions, thereby obtaining a porous support A supporting silver. The average particle diameter of the porous support A was 2.0 μm, and the silver ion content in the support with respect to the total mass of the porous support was equivalent to 0.5% by mass.

### Examples and Comparative Examples

The components synthesized in Synthesis Examples 1 to 3 or zeolite was added to and mixed with the curable composition prepared by the same method as in Examples 1 to 5 and Comparative Examples 1 to 3 described above according to the ratio (% by mass (wt %)) shown in Table 3, thereby preparing curable compositions for forming a hydrophilic processed portion. The ratio (% by mass) means the content of each component with respect to the total mass of the hydrophilic processed portion to be formed.

<Evaluation>

(1) Evaluation of Antibacterial Properties

A polycarbonate sheet (CARBOGLASS CFR110C manufactured by ASAHI GLASS CO., LTD.) was coated with the obtained curable composition for forming a hydrophilic processed portion. The composition was dried for 30 minutes at 60° C., and then the monomer was cured by UV irradiation. In this way, a hydrophilic processed portion was formed, and an evaluation sample (substrate with a hydrophilic processed portion) was prepared. The amount of the curable composition used for coating was adjusted such that the thickness of the hydrophilic processed portion (average thickness of the hydrophilic processed portion) became the thickness shown in Table 3.

(Evaluation of Initial Antibacterial Properties: Evaluation of Antibacterial Properties Exhibited within Short Time)

For evaluating the antibacterial properties (initial antibacterial properties), based on the evaluation method described in JIS Z 2801:2010, a test was performed by changing the contact time with the bacterial liquid to 1 hour from 24 hours. The bacterial count (bacteria/cm²) was measured after each test, and the initial antibacterial properties were evaluated according to the standards shown below. As bacterial species, E. coli was used. In order to check the validity of the test, a polycarbonate sheet (CARBOGLASS CFR110C manufactured by ASAHI GLASS CO., LTD.) not provided with a hydrophilic processed portion was also evaluated. As a result, it was confirmed that the bacterial count (bacteria/cm²) after an hour of contact was equal to or greater than 6.2×10² (bacteria/cm²). For practical use, “A” or “B” is preferable.

“A”: the bacterial count is less than 1/cm².

“B”: the bacterial count is equal to or greater than 1/cm² and less than 10/cm².

“C”: the bacterial count is equal to or greater than 10/cm².

(Evaluation of Antibacterial Properties after Durability Test)

In order to evaluate whether the evaluation sample prepared according to the aforementioned procedure has antibacterial properties that last over a long period of time, the following durability test was performed. Generally, in a case where contaminants adhere to the hydrophilic processed portion disposed on the surface of a member, the hydrophilic processed portion is wiped with a damp cloth in many cases. At this time, the elution of silver into water occurs and becomes the main cause of the deterioration of antibacterial properties. Accordingly, in a case where excellent antibacterial properties are exhibited even after the following abrasion test, it is understood that the antibacterial properties can be retained over a long period of time even after the hydrophilic processed portion is used.

During the durability test, the surface of the hydrophilic processed portion of the evaluation sample (substrate with a hydrophilic processed portion) prepared according to the aforementioned procedure was rubbed 36,000 times with a damp cloth under a load of 500 g. Then, the test for evaluating antibacterial properties was performed by the method described above, and the antibacterial properties were evaluated according to the following standards. As bacterial species, E. coli was used. As the damp cloth, a polyester cloth (trade name: ANTICON) immersed in pure water was used. For practical use, “A” or “B” is preferable.

“A”: the bacterial count was less than 1/cm².

“B” the bacterial count was equal to or greater than 1/cm² and less than 10/cm².

“C”: the bacterial count was equal to or greater than 10/cm².

“Zeolite” described in the column of “Porous support” in Table 3 means zeolite used in Synthesis Example 3 and does not support silver.

The water contact angle of the surface of the hydrophilic processed portion obtained in each of the examples was equal to or smaller than 40°.

Furthermore, the hydrophilic processed portion obtained in each of the examples included a porous support positioned such that a portion thereof protruded from the surface (flat surface) of the hydrophilic processed portion.

In addition, in Table 3, the average particle diameter is a value obtained by measuring 50% by volume cumulative diameter (D50) three times by using a laser diffraction/scattering-type particle size distribution measurement apparatus manufactured by HORIBA, Ltd, and calculating the average of the values obtained by the measurement performed three times.

(2) Evaluation of Antifogging Properties

The outer surface of the counting display portion 36 and the battery level display portion 37 in the portable radiographic imaging device 10 was coated with the obtained curable composition for forming a hydrophilic processed portion, followed by curing (ultraviolet irradiation treatment), thereby providing a hydrophilic processed portion containing an antibacterial agent and having undergone antibacterial processing. The average thickness of the hydrophilic processed portion was about 2 μm.

The water contact angle of the hydrophilic processed portion provided in the portable radiographic imaging devices obtained in the examples and the comparative examples was measured by the aforementioned method.

The evaluation method of the antifogging properties was the same as the evaluation method used in Examples 1 to 5 and Comparative Examples 1 to 3 described above.

The results are summarized in Table 3.

TABLE 3

| | | | | | Content with respect | | Average |
|---|---|---|---|---|---|---|---|
| | | | | | to total mass of | | thickness T |
| | Antibacterial agent | | Porous support | | hydrophilic processed | | of |
| | | Average | | Average | portion | | hydrophilic |
| | | particle | | particle diameter | Antibacterial | Porous | processed |
| | | diameter | | Db | agent | support | portion |
| | Type | Da (μm) | Type | (μm) | (wt %) | (wt %) | (μm) |
| Example 11 | Antibacterial | 0.9 | Porous support A | 2.0 | 1 | 0.5 | 3 |
| Example 12 | agent A | | | | 1.5 | 0.5 | 3 |
| Example 13 | | | | | 2 | 0.5 | 3 |
| Example 14 | | | | | 2.5 | 0.5 | 3 |
| Example 15 | | | | | 1.5 | 0.5 | 4 |
| Example 16 | | | | | 1.5 | 0.5 | 6 |
| Comparative | | | | | 1.5 | 0.5 | 8 |
| Example 8 | | | | | | | |
| Comparative | Antibacterial | 0.9 | — | — | 0.5 | 0 | 3 |
| Example 7 | agent A | | | | | | |
| Example 17 | Antibacterial | 0.9 | Porous support A | 2.0 | 1.5 | 0.01 | 3 |
| Example 18 | agent A | | | | 1.5 | 0.04 | 3 |
| Example 19 | | | | | 1.5 | 0.2 | 3 |
| Example 20 | | | | | 1.5 | 0.8 | 3 |
| Example 21 | | | | | 1.5 | 1 | 3 |
| Example 22 | Antibacterial | 0.5 | Porous support A | 2.0 | 1 | 0.5 | 3 |
| Example 23 | agent B | | | | 1.5 | 0.5 | 3 |
| Example 24 | | | | | 2 | 0.5 | 3 |
| Example 25 | | | | | 2.5 | 0.5 | 3 |
| Example 26 | Antibacterial | 0.9 | Zeolite | 2.0 | 1 | 0.5 | 3 |
| Example 27 | agent A | | | | 1.5 | 0.5 | 3 |
| Example 28 | | | | | 2 | 0.5 | 3 |

| | | | | | Evaluation | | |
|---|---|---|---|---|---|---|---|
| | | | | Water contact | Evaluation of | Evaluation of initial | Evaluation of |
| | | | | angle | antifogging | antibacterial | antibacterial properties |
| | T/Da | T/Db | Db/Da | (°) | properties | properties | after durability test |
| Example 11 | 3.3 | 1.5 | 2.2 | 10 | A | A | B |
| Example 12 | 3.3 | 1.5 | | 10 | A | A | A |
| Example 13 | 3.3 | 1.5 | | 10 | A | A | A |
| Example 14 | 3.3 | 1.5 | | 10 | A | A | A |
| Example 15 | 4.4 | 2 | | 10 | A | A | A |
| Example 16 | 6.7 | 3 | | 10 | A | A | B |
| Comparative | 8.9 | 4 | | 10 | A | A | C |
| Example 8 | | | | | | | |
| Comparative | 3.3 | — | — | 10 | A | B | C |
| Example 7 | | | | | | | |
| Example 17 | 3.3 | 1.5 | 2.2 | 10 | A | A | A |
| Example 18 | 3.3 | 1.5 | | 10 | A | A | A |
| Example 19 | 3.3 | 1.5 | | 10 | A | A | A |
| Example 20 | 3.3 | 1.5 | | 10 | A | B | B |
| Example 21 | 3.3 | 1.5 | | 10 | A | B | B |
| Example 22 | 6.0 | 1.5 | 4.0 | 10 | A | B | B |
| Example 23 | 6.0 | 1.5 | | 10 | A | B | B |
| Example 24 | 6.0 | 1.5 | | 10 | A | B | B |
| Example 25 | 6.0 | 1.5 | | 10 | A | B | B |
| Example 26 | 3.3 | 1.5 | 2.2 | 10 | A | B | B |
| Example 27 | 3.3 | 1.5 | | 10 | A | B | B |
| Example 28 | 3.3 | 1.5 | | 10 | A | B | B |

As shown in Table 3, it was confirmed that the hydrophilic processed portion in the substrate with a hydrophilic processed portion of the present invention has sufficient antifogging properties, exhibits antibacterial properties within a short time, and can retain the antibacterial properties over a long period of time.

Particularly, from the comparison between Example 11 and Example 12, it was confirmed that in a case where the content of the antibacterial agent is greater than 1.0% by mass, the result of the evaluation of antibacterial properties after the durability test becomes better.

From the comparison between Example 15 and Example 16, it was confirmed that in a case where the ratio (T/Da) is equal to or lower than 6.0, the result of the evaluation of antibacterial properties after the durability test becomes better.

From the comparison between Example 19 and Example 20, it was confirmed that in a case where the content of the porous support is equal to or smaller than 0.5% by mass, the results of the evaluation of initial antibacterial properties and the evaluation of antibacterial properties after the durability test become better.

From the comparison between Example 12 and Example 23, it was confirmed that in a case where the ratio (Db/Da) is equal to or lower than 3.5, the results of the evaluation of initial antibacterial properties and the evaluation of antibacterial properties after the durability test become better.

From the comparison between Example 12 and Example 27, it was confirmed that in a case where silver is supported on the porous support, the results of the evaluation of initial antibacterial properties and the evaluation of antibacterial properties after the durability test become better.

It was confirmed that Comparative Examples 7 and 8 in which the antifogging properties fall into a predetermined range have antifogging properties. However, regarding the evaluation of antibacterial properties, in a case where the porous support was not used as in Comparative Example 7, or in a case where the relationship of Formula (5) was not satisfied as in Comparative Example 8, excellent effects were not obtained.

The above results clearly show the effects of Embodiment 3 of the present invention.

### EXPLANATION OF REFERENCES

10: portable radiographic imaging device
12: radiation detector
13: control board
18: housing
19: irradiation surface
19A: imaging surface
20: TFT board
21: scintillator
22: light screen element
23: connector
24: connector
25: scan signal control circuit
26: signal detection circuit
27: connector
28: flexible cable
29: connector
30: flexible cable
31: front panel
32: back panel
33: top panel
34: holding portion
35: LED lamp
36: counting display portion
37: battery level display portion
38: inclined portion
40: substrate with hydrophilic processed portion
42: substrate
44: hydrophilic processed portion
50: hydrophilic processed sheet
52: main sheet
54: hydrophilic processed portion
56: pressure sensitive adhesive layer
58: release sheet
60, 60A, 60B: base with (hydrophilic processed) antibacterial film
62: base
64: hydrophilic portion
66: hydrophilic processed antibacterial film
70: substrate with hydrophilic processed portion
72: substrate
74: hydrophilic processed portion
80: substrate with hydrophilic processed portion
82: substrate
84: hydrophilic processed portion
86: antibacterial agent
88: silver
90: porous support which can adsorb silver ions
92: silver ion
100: portable radiographic imaging device (electronic cassette-type imaging device)
102: housing
104: radiation detector
106: body member
108: first cap member
112: battery mounting portion
114: connector connecting portion
116: irradiation surface
118: display portion
120: mammography device
122: face guard
124: breast support
126: breast compression plate
128: display portion
130: radiographic diagnostic device
132: imaging board
134, 136: grip
138: operation panel portion
140: medical mask
142: inner surface of mask
150: medical goggle
152: outer surface of goggle
160: transparent sterile medical bag
162: inner surface of sterile bag
164: tablet PC

What is claimed is:

1. A medical instrument comprising:
a hydrophilic processed portion on at least a portion of an outer surface of the instrument,
wherein the hydrophilic processed portion contains a hydrophilic polymer and an antibacterial agent,
a water contact angle of a surface of the hydrophilic processed portion is equal to or smaller than 30°,
provided that a silver content per unit area in the hydrophilic processed portion is P and a silver ion amount per unit area measured by an extraction test described below is Q, P and Q satisfy relationships of Formula (1) and Formula (2),
6.0≤P/Q Formula (1)
15.0≤Q Formula (2)
a unit of P is ng/cm²,
a unit of Q is ng/cm², and
the extraction test is a test in which a 1/500 normal nutrient broth medium specified in JIS Z 2801:2010 is used as an extractant; a temperature of the extractant is controlled within a range of 35±1° C.; the extractant is brought into contact with the hydrophilic processed portion for 1 hour; the silver ion amount extracted into the extractant is measured; and the obtained value is divided by a contact area between the hydrophilic processed portion and the extractant, thereby obtaining the silver ion amount Q per unit area.

2. The medical instrument according to claim 1,
wherein Q satisfies a relationship of Formula (3),
15.0≤Q≤25.0 Formula (3)

3. The medical instrument according to claim 1,
wherein the antibacterial agent includes a first antibacterial agent containing silver and a second antibacterial agent containing silver different from the first antibacterial agent.

4. The medical instrument according to claim 3,
wherein the first antibacterial agent contains silver and one support selected from the group consisting of calcium zinc phosphate and calcium phosphate, and
the second antibacterial agent contains silver and a support formed of zeolite.

5. A medical instrument comprising:
a hydrophilic processed portion on at least a portion of an outer surface of the instrument,
wherein the hydrophilic processed portion contains a hydrophilic polymer and at least one kind of antibacterial agent,
a water contact angle of a surface of the hydrophilic processed portion is equal to or smaller than 30°,
the antibacterial agent contains silver,
a silver ion amount per unit area measured by an extraction test described below is 15 to 50 ng/cm²,
in the extraction test, a 1/500 normal nutrient broth medium specified in JIS Z 2801:2010 is used as an extractant; a temperature of the extractant is controlled within a range of 35±1° C.; the extractant is brought into contact with the hydrophilic processed portion for 1 hour; the silver ion amount extracted into the extractant is measured; and the obtained value is divided by a contact area between the hydrophilic processed portion and the extractant, thereby obtaining the silver ion amount per unit area,
a unit of the silver ion amount is ng,
a unit of the contact area is cm², and
a unit of the silver ion amount per unit area is ng/cm².

6. The medical instrument according to claim 5,
wherein the antibacterial agent is a silver-supporting support containing a support and silver supported on the support.

7. A medical instrument comprising:
a hydrophilic processed portion on at least a portion of an outer surface of the instrument,
wherein the hydrophilic processed portion contains a hydrophilic polymer, an antibacterial agent containing silver, and a porous support which can adsorb silver ions,
a water contact angle of a surface of the hydrophilic processed portion is equal to or smaller than 30°,
provided that an average particle diameter of the antibacterial agent is Da, an average particle diameter of the porous support is Db, and an average thickness of the hydrophilic processed portion is T, Da, Db, and T satisfy relationships shown below,
T/Da>3.0 Formula (4)
T/Db≤3.0 Formula (5)
a unit of Da, Db, and T is μm.

8. The medical instrument according to claim 7,
wherein silver is supported on the porous support.

9. The medical instrument according to claim 7,
wherein Da and Db satisfy a relationship shown below,
Db/Da≤3.5 Formula (6)

10. The medical instrument according to claim 7,
wherein a content of the porous support with respect to a total mass of the hydrophilic processed portion is equal to or smaller than 0.5% by mass.

11. The medical instrument according to claim 7,
wherein the antibacterial agent contains silver and one support selected from the group consisting of calcium zinc phosphate and calcium phosphate, and
the porous support contains silver and a support formed of zeolite.

12. The medical instrument according to claim 1,
wherein a surface roughness Ra of the surface of the hydrophilic processed portion is 2 to 15 μm.

13. The medical instrument according to claim 1,
wherein the hydrophilic processed portion contains lubricant particles having an average particle diameter of 6 to 10 μm.

14. The medical instrument according to claim 1,
wherein an average thickness of the hydrophilic processed portion is 1 to 10 μm.

15. The medical instrument according to claim 1,
wherein a content of the antibacterial agent with respect to a total mass of the hydrophilic processed portion is 0.001% to 5% by mass.

16. The medical instrument according to claim 1,
wherein the antibacterial agent contains at least one kind of particles selected from the group consisting of ceramic particles supporting silver and silver particles.

17. The medical instrument according to claim 1 that is a radiographic imaging device.

18. The medical instrument according to claim 17,
wherein in the radiographic imaging device, the hydrophilic processed portion is provided on at least a surface that comes into contact with a subject at the time of imaging.

19. The medical instrument according to claim 17,
wherein the radiographic imaging device is a portable radiographic imaging device, and
the hydrophilic processed portion is provided on at least a surface on a side irradiated with radiation in a housing of the portable radiographic imaging device.

20. The medical instrument according to claim 19,
wherein the hydrophilic processed portion is further provided in a rear surface portion of the housing and/or a lateral surface portion of the housing.

21. The medical instrument according to claim 19,
wherein the portable radiographic imaging device as the radiographic imaging device has a housing body in the form of a cylinder having at least one open end, and
the housing body constitutes the housing by closing the one open end with a cap member.

22. The medical instrument according to claim 19,
wherein in the portable radiographic imaging device as the radiographic imaging device, at least a portion of the rear surface portion of the housing and at least a portion of the lateral surface portion of the housing are integrally formed.

23. The medical instrument according to claim 17,
wherein the radiographic imaging device is a mammography device, and
the hydrophilic processed portion is provided on at least a surface coming into contact with a subject within a surface of a face guard portion.

24. The medical instrument according to claim 23,
wherein in the mammography device, the hydrophilic processed portion is provided on at least a surface coming into contact with a subject within a surface of a breast support or on at least a surface coming into contact with a subject within a surface of a breast compression plate.

25. The medical instrument according to claim 1 that is a medical mask.

26. The medical instrument according to claim 1 that is a medical goggle.

27. The medical instrument according to claim 1 that is a transparent sterile medical bag.
